SEQUENCE LISTING (8 sequences)

SEQ ID NO: 1 - protein, 14 aa, Helicobacter pylori
FPLKRHDKVDDLSK

SEQ ID NO: 2 - protein, 125 aa, Helicobacter pylori
  ref <308>: DATABASE ACCESSION NUMBER: NCBI/3IEC_H
  ref <309>: DATABASE ENTRY DATE: 2010-01-27
  ref <313>: RELEVANT RESIDUES IN SEQ ID NO: (1)..(125)
GPVDNNNNNGLKNSTEPIYAKVNKKKTGQVASPEEPIYTQVAKKVNAKIDRLNQIASGLGGVGQAAGFPLKRHDKVDDLSKVGLSASPEPIYATIDDLGGPFPLKRHDKVDDLSKVGRSRNQELA

SEQ ID NO: 3 - DNA, 3540 bp, Helicobacter pylori
  CDS (1)..(3540)
  ref <308>: DATABASE ACCESSION NUMBER: NCBI/AF247651
  ref <309>: DATABASE ENTRY DATE: 2000-04-13
  ref <313>: RELEVANT RESIDUES IN SEQ ID NO: (1)..(3540)
atgactaacgaaaccattaaccaacaaccacaaaccgaagcggcttttaacccgcagcaatttatcaataatcttcaagtggcttttcttaaagttgataacgctgtcgcttcatacgatcctgatcaaaaaccaatcattgataagaacgatagggataacaggcaagcttttgagggaatctcgcaattaagggaagaatactccaataaagcgatcaaaaatcctaccaaaaagaatcagtatttttcagactttatcaataagagcaatgatttaatcaacaaagacgatctcattgatgtagaatcttccacaaagagctttcagaaatttggggatcagcgttaccgaattttcacaagttgggtgtcccatcaaaacgatccgtctaaaatcaacacccgatcgatccgaaattttatggaaaatatcatacaaccccctatccctgatgacaaagaaaaagcagagtttttgaaatctgccaaacaatcttttgcaggaatcattatagggaatcaaatccgaacggatcaaaagttcatgggcgtgtttgatgaatccttgaaagaaaggcaagaagcagaaaaaaatggagggcctactggtggggattggttggatatttttctctcatttatatttgacaaaaaacaatcttctgatgtcaaagaagcaatcaatcaagaaccagttccccatgtccaaccagatatagccactaccaccacccacatacaaggcttaccgcctgaatctagggatttgcttgatgaaaggggtaatttttctaaattcactcttggcgatatggaaatgttagatgttgagggagtcgctgacattgatcccaattacaagttcaatcaattattgattcacaataacgctctgtcttctgtgttaatggggagtcataatggcatagaacctgaaaaagtttcattattgtatgcgggcaatggtggtttcggagccaagcacgattggaacgccaccgttggttataaagaccaacaaggtaacaatgtggctacaataattaatgtgcatatgaaaaacggcagtggcttggtcatagcaggtggtgagaaagggattaataaccctagtttttatctctacaaagaagaccaactcacaggctcacaacgagcattgagtcaagaagagatccgaaacaaaatagatttcatggaatttcttgcacaaaataatgctaaattagacaacttgagcgagaaagagaaagaaaaattccgaactgagattaaggatttccaaaaagactctaaggcttatttagacgccctagggaatgatcgtattgcttttgtttctaaaaaagacccaaaacattcagctttaattactgagtttggtaagggggatttgagctacactctcaaagattatgggaaaaaagcagataaagctttagatagggagaaaaatgttactcttcaaggtagcctaaaacatgatggcgtgatgtttgttgattattctaatttcaaatacaccaacgcctccaagaatcccaataagggtgtaggcgttacaaatggcgtttcccatttagaagcaggctttagcaaggtagctgtctttaatttgcctgatttaaataatctcgctatcactagtttcgtaaggcggaatttagaggataaactagccactaaaggattgtccctacaagaagctaataagcttatcaaagattttttgagcagcaacaaagaattggttggaaaagctttaaacttcaataaagctgtagctgaagctaaaaacacaggcaattatgatgaagtgaaaaaagctcagaaagatcttgaaaaatctctaaggaaacgagagcatttagagaaagaagtagagaaaaaattggagagcaaaagcggcaacaaaaacaaaatggaagcgaaatctcaagctaacagccaaaaagatgagatttttgcgttgattaataaagaggctaatagggacgcaagagcaatcgcttacgctacgaatcttagaggcgtcaaaagggaattgtctgataaacttgaaaatgtcaacaagaatttgaaagactttagtaaatcttttgatgaattcaaaaatggcaaaaataaggatttcagcaaggcagaagaaacgctaaaaacccttaaaggctcggtgaaagatttaggtatcaatccagaatggatttcaaaagttgaaaaccttaatgcagctttgaatgaattcaaaaatggcaaaaataaggatttcagcaaggtaacgcaagcaaaaagcgaccttgaaaattccgttaaagatgtgatcatcaatcaaaagataacggataaagttgacaatctcaatcaagcggtatcagtggctaaagcaacgggtgatttcagtagggtagagcaagcgttagccgatctcaaaaacttctcaaaggagcaattggcccaacaagctcaaaaaaatgaagatttcaatactggaaaaaattctgcactataccaatccgttaagaatggtgtgaatggaaccctagtcggtaatgggttatctaaagcagaagccacaactctttctaaaaacttttcggacatcaagaaagagttgaatgcaaaattggggaatttcaataacaataacaataatggactcaaaaacgaacccatttacgctcaagttaataaaaagaaagcaggacaagtagttagccctgaagagcccatttacgctcaagttgctaaaaaggtaaatgcaaaaattgaccaactcaatcaagcggcaagtggtttgggtggtgtagggcaagcgggcttccctttgaaaaggcatgataaagttgatgatctcagtaaggtagggcgatcggttagccctgaacccatttatgctacgattgatgatctcggcggacctttccctttgaaaaagcatgctaaagttgaagatctcagtaaggtagggctttcaagggagcaagaattgactcagaaaattgacaatctcaatcaagcggtatcagaagctaaagcaggtttttttggcaatctagaacaaacgatagacaatctcaaagattctacaaaacacaatcccatgaatctatgggctgaaagtgcaaaaaaagtgcctgctagtttgtcagcgaaactagacaattacgctactaacagccacacacgcattaatagcaatatccaaaatggagcaatcaatgaaaaagcgaccggtatgctaacgcaaaaaaaccctgagtggctcaagctcgtgaatgataagatcgttgtgcataatgtaggaagcgttcctttgtcagagtatgataaaattggcttcaaccagaagaatatgaaagattattctgattcgttcaagttttccaccaagttgaacaatgctgtaaaagatgttaggtctggctttacgcaatttttagccaatgcattttctacaggatattactgcttggcggaagaaaatgcagagcatggaatcgaaaatgttaatacaaaaggtggtttccaaaaatcttaa

SEQ ID NO: 4 - protein, 1179 aa, Helicobacter pylori
MTNETINQQPQTEAAFNPQQFINNLQVAFLKVDNAVASYDPDQKPIIDKNDRDNRQAFEGISQLREEYSNKAIKNPTKKNQYFSDFINKSNDLINKDDLIDVESSTKSFQKFGDQRYRIFTSWVSHQNDPSKINTRSIRNFMENIIQPPIPDDKEKAEFLKSAKQSFAGIIIGNQIRTDQKFMGVFDESLKERQEAEKNGGPTGGDWLDIFLSFIFDKKQSSDVKEAINQEPVPHVQPDIATTTTHIQGLPPESRDLLDERGNFSKFTLGDMEMLDVEGVADIDPNYKFNQLLIHNNALSSVLMGSHNGIEPEKVSLLYAGNGGFGAKHDWNATVGYKDQQGNNVATIINVHMKNGSGLVIAGGEKGINNPSFYLYKEDQLTGSQRALSQEEIRNKIDFMEFLAQNNAKLDNLSEKEKEKFRTEIKDFQKDSKAYLDALGNDRIAFVSKKDPKHSALITEFGKGDLSYTLKDYGKKADKALDREKNVTLQGSLKHDGVMFVDYSNFKYTNASKNPNKGVGVTNGVSHLEAGFSKVAVFNLPDLNNLAITSFVRRNLEDKLATKGLSLQEANKLIKDFLSSNKELVGKALNFNKAVAEAKNTGNYDEVKKAQKDLEKSLRKREHLEKEVEKKLESKSGNKNKMEAKSQANSQKDEIFALINKEANRDARAIAYATNLRGVKRELSDKLENVNKNLKDFSKSFDEFKNGKNKDFSKAEETLKTLKGSVKDLGINPEWISKVENLNAALNEFKNGKNKDFSKVTQAKSDLENSVKDVIINQKITDKVDNLNQAVSVAKATGDFSRVEQALADLKNFSKEQLAQQAQKNEDFNTGKNSALYQSVKNGVNGTLVGNGLSKAEATTLSKNFSDIKKELNAKLGNFNNNNNNGLKNEPIYAQVNKKKAGQVVSPEEPIYAQVAKKVNAKIDQLNQAASGLGGVGQAGFPLKRHDKVDDLSKVGRSVSPEPIYATIDDLGGPFPLKKHAKVEDLSKVGLSREQELTQKIDNLNQAVSEAKAGFFGNLEQTIDNLKDSTKHNPMNLWAESAKKVPASLSAKLDNYATNSHTRINSNIQNGAINEKATGMLTQKNPEWLKLVNDKIVVHNVGSVPLSEYDKIGFNQKNMKDYSDSFKFSTKLNNAVKDVRSGFTQFLANAFSTGYYCLAEENAEHGIENVNTKGGFQKS

SEQ ID NO: 5 - DNA, 42 bp, Helicobacter pylori
ttccctttgaaaaggcatgataaagttgatgatctcagtaag

SEQ ID NO: 6 - protein, 4 aa, Artificial Sequence
  linker
GGSG

SEQ ID NO: 7 - protein, 4 aa, Artificial Sequence
  linker
SAGG

SEQ ID NO: 8 - protein, 5 aa, Artificial Sequence
  linker
GGGGS

(19) United States
(12) Patent Application Publication
(10) Pub. No.: US 2013/0331330 A1
(43) Pub. Date: Dec. 12, 2013

(54) METHODS OF TREATING ALZHEIMER'S DISEASE AND OTHER TAUOPATHIES WITH INHIBITORS OF MICROTUBULE AFFINITY REGULATING KINASE

(71) Applicant: The board of Trustees of the Leland Stanford Junior University, Palo Alto, CA (US)

(72) Inventors: Bingwei Lu, Stanford, CA (US); Jayakumar Rajadas, Cupertino, CA (US); Dhananjay Wagh, Fremont, CA (US); Wendou Yu, Salt Lake City, UT (US)

(21) Appl. No.: 13/913,364

(22) Filed: Jun. 7, 2013

Related U.S. Application Data
(60) Provisional application No. 61/657,613, filed on Jun. 8, 2012.

(51) Int. Cl. A61K 38/16; A61K 31/7088; A61K 38/10

(52) U.S. Cl. CPC A61K 38/164; A61K 38/10; A61K 31/7088
USPC 514/17.8; 514/17.7; 514/44 R

(57) ABSTRACT

Methods of treating Alzheimer's disease and other tauopathies are disclosed. In particular, the invention relates to methods of treating Alzheimer's disease and other tauopathies with inhibitors of microtubule affinity regulating kinase (MARK). Hyperphosphorylation of tau by MARK is associated with the pathogenic deposition of intracellular neurofibrillary tangles and loss of synaptic markers, dendritic spines, and synapses during progression of Alzheimer's disease and other tauopathies. Peptide inhibitors derived from the Helicobacter pylori protein CagA block phosphorylation of tau in neuronal cells and thereby decrease the deposition of neurofibrillary tangles and the toxic effects of Abeta-42.

### CROSS-REFERENCE TO RELATED APPLICATION

This application claims benefit under 35 U.S.C. §119(e) of provisional application 61/657,613, filed Jun. 8, 2012, which application is hereby incorporated by reference in its entirety.

### TECHNICAL FIELD

The present invention pertains generally to methods of treating Alzheimer's disease and other tauopathies. In particular, the invention relates to methods of treating Alzheimer's disease and other tauopathies using inhibitors of microtubule affinity regulating kinase (MARK) that reduce hyperphosphorylation of tau in neuronal cells and deposition of neurofibrillary tangles.

### BACKGROUND

Alzheimer's disease (AD) is the leading cause of dementia in the elderly. Although it is well-known that disease progression is associated with formation of amyloid plaques (AP) and neurofibrillary tangles (NFT), memory deficits in AD patients do not correlate well with either AP or NFT burden. Rather, loss of synaptic markers better predicts clinical symptoms and disease progression and suggests that AD is a disease of synaptic failure (Selkoe (2002) Science 298:789-791). Synapses and dendritic spines are dynamic structures whose plasticity is thought to underlie learning and memory (Penzes et al. (2011) Nat. Neurosci. 14:285-293).

Accumulating evidence supports a role for amyloid-β (Aβ) as the causative agent in synaptic and spine pathology in AD (Spires et al. (2005) J. Neurosci. 25:7278-7287; Reddy et al. (2008) Trend. Mol. Med. 14:45-53; Selkoe (2008) Brain Res. 192:106-113). Rare genetic mutations cause familial AD by altering the production or metabolism of Aβ (Hardy et al. (2002) Science 297:353-356; Holtzman et al. (2011) Sci. Transl. Med. 3:77sr71), the soluble pool of which correlates with disease progression and severity (Selkoe (2008) Behav. Brain Res. 192:106-113). Depletion of soluble Aβ in mouse AD models rescued the cognitive deficits (Billings et al (2005) Neuron, 45:675-688). Moreover, synthetic Aβ oligomers or those purified from cultured cells or brain samples can induce neuritic degeneration, neurotransmission defects, and spine loss (Yankner et al. (1989) Science 245:417-420; Walsh et al. (2002) Nature 416:535-539; Jin et al. (2011) Proc. Natl. Acad. Sci. USA 108:5819-5824; Lambert et al. (1998) Proc. Natl. Acad. Sci. USA 95:6448-6453). The detailed cellular and biochemical mechanisms involved, however, remain poorly defined.

Recent studies have supported tau as a major mediator of Aβ toxicity. In mouse models, intracranial injection of Aβ or crossing an APP transgene into tau transgenic animals promoted NFT pathology (Lewis et al. (2001) Science 293:1487-1491; Gotz et al. (2001) Science 293:1491-1495; Oddo et al. (2003) Neuron 39:409-421), and antibody-based removal of Aβ reduced hyperphosphorylated tau and rescued behavioral and pathological defects in a APP/Psn/tau 3×Tg AD mouse model (Oddo et al. (2004) Neuron 43:321-332). Moreover, removal of tau relieved Aβ-induced neurotoxicity in culture (Rapoport et al. (2002) Proc. Natl. Acad. Sci. USA 99:6364-6369), and prevented Aβ-induced behavioral deficits in an h-APP Tg mouse model (Roberson et al. (2007) Science 316:750-754). Together, these studies support the critical involvement of tau in mediating the toxicity of Aβ in AD pathogenesis.

It is not clear how tau abnormality arises in AD, but may be linked to tau hyperphosphorylation (Spires-Jones et al. (2009) Trend. Neurosci. 32:150-159; Ittner et al. (2011) Nat. Rev. Neurosci. 12:65-72). A number of kinases and phosphatases are known to regulate tau phosphorylation (Lee et al. (2001) Annu. Rev. Neurosci. 24:1121-1159). Partitioning defective-1 (PAR-1)/microtubule affinity regulating kinase (MARK) was originally identified in C. elegans for its role in regulating cell polarity (Guo et al. (1995) Cell 81:611-620). PAR-1 and its mammalian homologue MARK phosphorylate tau and have been implicated in pathogenic deposition of neurofibrillary tangles (Drewes et al. (1997) Cell 89:297-308; Chin et al. (2000) J. Neuropathol. Exp. Neurol. 59:966-971). In Drosophila models, PAR-1-mediated phosphorylation is required for conferring tau toxicity (Nishimura et al. (2004) Cell 116:671-682). Elevation of PAR-1/MARK-mediated tau phosphorylation (at 12E8 sites) was observed in AD patients and mouse AD models (Augustinack et al. (2002) Acta. Neuropathol. 103:26-35; Perez et al. (2005) Neuroscience 130:339-347). Activated Drosophila PAR-1 also directly phosphorylates the postsynaptic density protein 95 (PSD-95) homologue discs large (Dlg), impairing its postsynaptic localization (Zhang et al. (2007) Neuron 53:201-215), which might be mechanistically related to the synaptic loss of PSD-95 seen in early stages of AD in patients and mouse models (Gylys et al. (2004) Am. J. Pathol. 165:1809-1817; Almeida et al. (2005) Neurobiol. Dis. 20:187-198).

Current treatments for Alzheimer's disease ameliorate symptoms, but fail to halt the progression of the disease. Thus, there remains a need for new treatments that interfere with or prevent the pathogenic steps that lead to progression of Alzheimer's disease.

### SUMMARY

The invention relates to methods of treating Alzheimer's disease and other tauopathies with inhibitors of microtubule affinity regulating kinase (MARK). Hyperphosphorylation of tau by MARK is associated with the pathogenic deposition of intracellular neurofibrillary tangles in the brain. Inhibitors of MARK block phosphorylation of tau in neuronal cells and thereby decrease the deposition of neurofibrillary tangles and the toxic effects of Abeta-42.

MARK inhibitors that can be used in the practice of the invention include CagA proteins, polypeptides, and peptides, and nucleic acids encoding them. In certain embodiments, the MARK inhibitor is selected from the group consisting of:
a) a peptide comprising the amino acid sequence of SEQ ID NO: 1;
b) a peptide comprising an amino acid sequence having at least 95% identity to the sequence of SEQ ID NO: 1, wherein the peptide inhibits the activity of MARK;
c) a polypeptide comprising the amino acid sequence of SEQ ID NO: 2;
d) a polypeptide comprising an amino acid sequence having at least 95% identity to the sequence of SEQ ID NO: 2, wherein the polypeptide inhibits the activity of MARK;
e) a polypeptide comprising the amino acid sequence of SEQ ID NO: 4;
f) a polypeptide comprising an amino acid sequence having at least 95% identity to the sequence of SEQ ID NO: 4, wherein the polypeptide inhibits the activity of MARK; and
g) a CagA peptide of 14 to 50 amino acids in length comprising at least residues 941 to 954 of the amino acid sequence of SEQ ID NO:4.

In other embodiments, the MARK inhibitor is a recombinant polynucleotide comprising a promoter operably linked to a polynucleotide encoding an inhibitor of MARK. The recombinant polynucleotide may comprise an expression vector, for example, a bacterial plasmid vector or a viral expression vector, such as, but not limited to, an adenovirus, retrovirus (e.g., γ-retrovirus and lentivirus), poxvirus, adeno-associated virus, baculovirus, or herpes simplex virus vector.

In certain embodiments, the recombinant polynucleotide expressing the inhibitor of MARK, comprises a polynucleotide selected from the group consisting of:
a) a polynucleotide encoding a peptide comprising the amino acid sequence of SEQ ID NO: 1;
b) a polynucleotide encoding a peptide comprising an amino acid sequence having at least 95% identity to the sequence of SEQ ID NO: 1, wherein the peptide inhibits the activity of MARK;
c) a polynucleotide encoding a polypeptide comprising the amino acid sequence of SEQ ID NO: 2;
d) a polynucleotide encoding a polypeptide comprising an amino acid sequence having at least 95% identity to the sequence of SEQ ID NO: 2, wherein the polypeptide inhibits the activity of MARK;
e) a polynucleotide encoding a polypeptide comprising the amino acid sequence of SEQ ID NO: 4;
f) a polynucleotide encoding a polypeptide comprising an amino acid sequence having at least 95% identity to the sequence of SEQ ID NO: 4, wherein the polypeptide inhibits the activity of MARK;
g) a polynucleotide encoding a CagA peptide of 14 to 50 amino acids in length, said peptide comprising at least residues 941 to 954 of the amino acid sequence of SEQ ID NO:4;
h) a CagA polynucleotide of 42 to 150 nucleotides in length comprising at least nucleotides 2821 to 2862 of the nucleotide sequence of SEQ ID NO:3;
i) a polynucleotide comprising the nucleotide sequence of SEQ ID NO:5; and
j) a polynucleotide comprising a nucleotide sequence having at least 95% identity to the sequence of SEQ ID NO:5, wherein the polynucleotide encodes a polypeptide that inhibits the activity of MARK.

In certain embodiments, the MARK inhibitor further comprises a tag, a detectable label, a cell penetrating peptide, a linker, or a signal sequence.

Tauopathies that can be treated by the methods of the invention include, but are not limited to, Alzheimer's disease, progressive supranuclear palsy, dementia pugilistica (chronic traumatic encephalopathy), frontotemporal dementia, frontotemporal lobar degeneration, parkinsonism linked to chromosome 17, lytico-Bodig disease (Parkinson-dementia complex of Guam), ganglioglioma, gangliocytoma, meningioangiomatosis, subacute sclerosing panencephalitis, lead encephalopathy, tuberous sclerosis, Hallervorden-Spatz disease, lipofuscinosis, Pick's disease, corticobasal degeneration, Argyrophilic grain disease (AGD), and tangle-predominant dementia.

In one aspect, the invention includes a method of treating a subject for a tauopathy, the method comprising administering a therapeutically effective amount of an inhibitor of microtubule affinity-regulating kinase (MARK) to the subject. By “therapeutically effective amount” is intended an amount that when administered, brings about a positive therapeutic response with respect to treatment of an individual for Alzheimer's disease or other tauopathy, such as an amount that that inhibits MARK and reduces tau or PSD-95 hyperphosphorylation. Additionally, a cycle of treatment with a MARK inhibitor may reduce or prevent deposition of neurofibrillary tangles, loss of expression of synaptic markers, loss of dendritic spines, or loss of synapses. Preferably, the individual undergoing treatment according to the invention exhibits an improvement in one or more symptoms of Alzheimer's disease or other tauopathy, including such improvements as improved memory and cognitive function. In certain embodiments, multiple therapeutically effective doses of compositions comprising one or more MARK inhibitors (e.g., CagA peptides or polypeptides or nucleic acids encoding CagA peptides or polypeptides) are administered.

In another aspect, the invention includes a method of inhibiting tau hyperphosphorylation in the brain of a subject, the method comprising administering an effective amount of an inhibitor of microtubule affinity-regulating kinase (MARK) to the subject. In certain embodiments, the MARK inhibitor is a CagA protein, polypeptide, or peptide. In other embodiments, the MARK inhibitor is a recombinant polynucleotide comprising a promoter operably linked to a polynucleotide encoding an inhibitor of MARK.

In another aspect, the invention includes a method of assessing the efficacy of a prophylactic treatment of a subject, the method comprising:
a) administering to a subject in need thereof a therapeutically effective amount of a MARK inhibitor; and
b) monitoring the subject for neurofibrillary tangles after administration of the MARK inhibitor.
In certain embodiments, the method further comprises monitoring the subject for tau or PSD-95 hyperphosphorylation, loss of expression of synaptic markers, loss of dendritic spines, or loss of synapses.

In another aspect, the invention includes a method of inhibiting MARK in a neuronal cell, the method comprising introducing an effective amount of a CagA inhibitor of MARK into the neuronal cell.

In another aspect, the invention includes a method of inhibiting MARK in a culture of neurons, the method comprising introducing an effective amount of a CagA inhibitor of MARK into the culture.

In another aspect, the invention includes a neuronal cell transformed with a recombinant polynucleotide comprising a promoter operably linked to a polynucleotide encoding an inhibitor of MARK.

In another aspect, the invention includes a method for inhibiting the phosphorylation of tau in a neuronal cell, the method comprising:
a) transforming a neuronal cell with a recombinant polynucleotide encoding a CagA polypeptide or a peptide fragment thereof that inhibits MARK;
b) culturing the transformed neuronal cell under conditions whereby said CagA polypeptide or peptide fragment is expressed.

In another aspect, the invention includes a method of preparing a pharmaceutical composition comprising a MARK inhibitor, the method comprising dissolving the MARK inhibitor in a pharmaceutically acceptable solvent and spray drying to produce a powder. In one embodiment, the powder comprises spherical particles ranging from about 500 nm to about 5 μm in diameter.

In another aspect, the invention includes a kit comprising a pharmaceutical composition comprising a MARK inhibitor and instructions for treating a tauopathy. The MARK inhibitor may be a CagA protein, polypeptide, or peptide, or a nucleic acid encoding a CagA protein, polypeptide, or peptide. The kit may further comprise means for delivering the composition to a subject.

These and other embodiments of the subject invention will readily occur to those of skill in the art in view of the disclosure herein.

### BRIEF DESCRIPTION OF THE FIGURES

FIGS. 1A-1C show that overexpression of MARK4 causes synaptic and spine abnormalities in rat hippocampal neurons. FIGS. 1A-1C show the effects of overexpression of wild-type (WT) MARK4 or the kinase-dead (KD) form of MARK4 on postsynaptic PSD-95 clusters (FIG. 1A), AMPA receptor GluR1 clusters (FIG. 1B), presynaptic Synapsin I clusters (FIG. 1C), and spine numbers (FIGS. 1A-1C). Data quantification is shown in FIG. 1F. FIG. 1D shows the differential response of PSD-95-S561A and PSD-95-WT synaptic localization to MARK4-WT over-expression. FIG. 1E shows the effect of h-tau-WT or h-tau-S2A co-expression on MARK4-WT overexpression-induced spines loss. FIG. 1F shows the quantification of spine number and PSD-95 (FIG. 1A), GluR1 (FIG. 1B) and Synapsin I (FIG. 1C) clusters in neurons transfected with MARK4-WT or MARK4-KD (***P<0.001 in Student's t-test). FIG. 1G (left side) shows the quantification of PSD-95 clusters in neurons co-transfected with MARK4-WT or MARK4-KD and EGFP-tagged PSD-95-WT or PSD-95-S561A (***P<0.001 in Student's t-test). FIG. 1G (right side) shows the quantification of spine number in neurons co-transfected with MARK4-WT or MARK4-KD and h-tau-WT or h-tau-S2A (***P<0.001 in Student's t-test). Scale bar, 5 μm.

FIGS. 2A-2H show that synaptic and spine toxicity is induced by Aβ oligomers and Aβ-induced spine loss can be rescued by h-tau-S2A. FIG. 2A shows that Aβ treatment increased endogenous tau phosphorylation at the 12E8 sites. Hippocampal neurons at 13 days in vitro were treated with Aβ oligomers for 12 hours and used for western blot analysis. Actin served as a loading control. FIGS. 2B-2E show the effects of Aβ treatment on PSD-95 clusters (FIG. 2C), GluR1 clusters (FIG. 2D), Synapsin I clusters (FIG. 2E) and spine number (FIG. 2B) in EGFP-transfected neurons. Data quantification is shown in FIG. 2B (***P<0.001 in Student's t-test). FIGS. 2F-2H shows the resistance of h-tau-S2A-transfected neurons to Aβ-induced loss of spines (FIG. 2F and FIG. 2G) and PSD-95 and GluR1 clusters (FIG. 2H). Scale bar, 10 μm.

FIGS. 3A-3E show that the MARK peptide inhibitor (MKI) inhibits PAR-1/MARK-mediated tau phosphorylation and MARK4 overexpression-induced synaptic and spine toxicity. FIG. 3A shows that MKI-EGFP inhibited phosphorylation of endogenous tau as assessed by western blot analysis using phospho-specific antibodies 12E8 and PHF-1 (FIG. 3A). FIG. 3B shows that MKI-EGFP inhibited phosphorylation of HA-tagged exogenous h-tau-WT at the 12E8 sites. Robust 12E8 staining (gray) is seen in EGFP-transfected control neurons. Total tau is stained with anti-HA (gray). FIG. 3C shows that MKI-EGFP has no effect on AMPK kinase activity as detected with the phospho-acetyl-CoA carboxylase (Ser79) (pACC) antibody. EGFP-transfected neurons served as control. FIGS. 3D and 3E show that MKI-EGFP blocked MARK4-overexpression-induced loss of spines (FIGS. 3D and 3E) and PSD-95 and GluR1 clusters (FIG. 3E). Data quantification is shown in (3E) (***P<0.001 in Student's t-test). Scale bar, 10 μm.

FIGS. 4A-4D show that MKI rescues Aβ-induced synaptic and dendritic spine defects at the morphological and electrophysiological levels. FIGS. 4A-4C show the effects of MKI-EGFP on Aβ-induced loss of PSD-95 (FIG. 4A) and GluR1 (FIG. 4B) clusters and spine numbers. Neurons transfected with MKI-EGFP or EGFP were treated with Aβ oligomer or mock-treated with solvent as control. The cytoplasmic GFP signals allowed detection of dendritic spines, and PSD-95 or GluR1 clusters were detected by immunostaining Quantification of data is shown in (FIG. 4C) (***P<0.001 in Student's t-test). FIG. 4D shows sample traces of synaptic activity (mEPSCs) recorded in neurons from four different conditions (control-EGFP, Aβ, MKI-EGFP+Aβ and MKI-EGFP alone) held at −70 mV. Summary graphs from four to six culture sets plotting normalized mEPSC frequency and mEPSC amplitude are shown beneath the traces. Scale bar, 5 μm.

FIG. 5 shows that overexpression of MARK4 in rat hippocampal neurons leads to increased phosphorylation of endogenous tau at the 12E8 epitope sites. Rat hippocampal neurons transfected with EGFP tagged MARK4-WT or MARK4-KD were immunostained with anti-EGFP (gray) and 12E8 (gray) antibodies. The merged images are shown to the left. Boxed areas are shown at higher magnification (3×) below each panel. Overexpression of MARK4-WT leads to increased phosphorylation of endogenous tau at the 12E8 sites, compared to overexpression of MARK4-KD, which does not affect tau phosphorylation. Scale bar: 15 μm in low-magnification panels, 5 μm in high-magnification panels.

### DETAILED DESCRIPTION

The practice of the present invention will employ, unless otherwise indicated, conventional methods of pharmacology, chemistry, biochemistry, recombinant DNA techniques and immunology, within the skill of the art. Such techniques are explained fully in the literature. See, e.g., Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook et al., Molecular Cloning: A Laboratory Manual (3^{rd} Edition, 2001); Methods In Enzymology (S. Colowick and N. Kaplan eds., Academic Press, Inc.); Fmoc Solid Phase Peptide Synthesis: A Practical Approach (W. C. Chan and Peter D. White eds., Oxford University Press, 1^{st} edition, 2000); N. Leo Benoiton, Chemistry of Peptide Synthesis (CRC Press; 1^{st} edition, 2005); Peptide Synthesis and Applications (Methods in Molecular Biology, John Howl ed., Humana Press, 1^{st} ed., 2005); and Pharmaceutical Formulation Development of Peptides and Proteins (The Taylor & Francis Series in Pharmaceutical Sciences, Lars Hovgaard, Sven Frokjaer, and Marco van de Weert eds., CRC Press; 1^{st} edition, 1999).

All publications, patents and patent applications cited herein, whether supra or infra, are hereby incorporated by reference in their entireties.

### I. Definitions

In describing the present invention, the following terms will be employed, and are intended to be defined as indicated below.

It must be noted that, as used in this specification and the appended claims, the singular forms “a,” “an” and “the” include plural referents unless the content clearly dictates otherwise. Thus, for example, reference to “a MARK inhibitor” includes a mixture of two or more MARK inhibitors, and the like.

The term “about,” particularly in reference to a given quantity, is meant to encompass deviations of plus or minus five percent.

The terms “peptide”, “oligopeptide” and “polypeptide” refer to any compound comprising naturally occurring or synthetic amino acid polymers or amino acid-like molecules including but not limited to compounds comprising amino and/or imino molecules. No particular size is implied by use of the terms “peptide”, “oligopeptide” or “polypeptide” and these terms are used interchangeably. Included within the definition are, for example, polypeptides containing one or more analogs of an amino acid (including, for example, unnatural amino acids, etc.), polypeptides with substituted linkages, as well as other modifications known in the art, both naturally occurring and non-naturally occurring (e.g., synthetic). Thus, synthetic oligopeptides, dimers, multimers (e.g., tandem repeats, linearly-linked peptides), cyclized, branched molecules and the like, are included within the definition. The terms also include molecules comprising one or more peptoids (e.g., N-substituted glycine residues) and other synthetic amino acids or peptides. (See, e.g., U.S. Pat. Nos. 5,831,005; 5,877,278; and 5,977,301; Nguyen et al. (2000) Chem. Biol. 7(7):463-473; and Simon et al. (1992) Proc. Natl. Acad. Sci. USA 89(20):9367-9371 for descriptions of peptoids). Non-limiting lengths of peptides suitable for use in the present invention includes peptides of 3 to 5 residues in length, 6 to 10 residues in length (or any integer therebetween), 11 to 20 residues in length (or any integer therebetween), 21 to 75 residues in length (or any integer therebetween), 75 to 100 (or any integer therebetween), or polypeptides of greater than 100 residues in length. Typically, polypeptides useful in this invention can have a maximum length suitable for the intended application. Preferably, the polypeptide is between about 3 and 100 residues in length. Generally, one skilled in art can easily select the maximum length in view of the teachings herein. Further, peptides and polypeptides, as described herein, for example synthetic peptides, may include additional molecules such as labels or other chemical moieties. Such moieties may further enhance interaction of the peptides or polypeptides with MARK and/or further detection of the peptides or polypeptides.

Thus, references to polypeptides or peptides also include derivatives of the amino acid sequences of the invention including one or more non-naturally occurring amino acids. A first polypeptide or peptide is “derived from” a second polypeptide or peptide if it is (i) encoded by a first polynucleotide derived from a second polynucleotide encoding the second polypeptide or peptide, or (ii) displays sequence identity to the second polypeptide or peptide as described herein. Sequence (or percent) identity can be determined as described below. Preferably, derivatives exhibit at least about 50% percent identity, more preferably at least about 80%, and even more preferably between about 85% and 99% (or any value therebetween) to the sequence from which they were derived. Such derivatives can include postexpression modifications of the polypeptide or peptide, for example, glycosylation, acetylation, phosphorylation, and the like.

Amino acid derivatives can also include modifications to the native sequence, such as deletions, additions and substitutions (generally conservative in nature), so long as the polypeptide or peptide maintains the desired activity (e.g., inhibits MARK activity). These modifications may be deliberate, as through site-directed mutagenesis, or may be accidental, such as through mutations of hosts that produce the proteins or errors due to PCR amplification. Furthermore, modifications may be made that have one or more of the following effects: increasing affinity and/or specificity for MARK and facilitating cell processing. Polypeptides and peptides described herein can be made recombinantly, synthetically, or in tissue culture.

A CagA polynucleotide, nucleic acid, oligonucleotide, protein, polypeptide, or peptide refers to a molecule derived from Helicobacter pylori. The molecule need not be physically derived from Helicobacter pylori, but may be synthetically or recombinantly produced. A number of CagA nucleic acid and protein sequences are known. Representative CagA sequences are presented in SEQ ID NOS:1-5 and additional representative sequences are listed in the National Center for Biotechnology Information (NCBI) database. See, for example, NCBI entries: Accession Nos. AF247651, JQ318032, JQ318035, JQ318033, GU212666, GU212664, GU212662, GU212660, GU212658, GU212665, and GU212663; all of which sequences (as entered by the date of filing of this application) are herein incorporated by reference. Any of these sequences or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, can be used to construct a CagA peptide or polypeptide, or a nucleic acid encoding a CagA peptide or polypeptide, as described herein.

By “fragment” is intended a molecule consisting of only a part of the intact full length sequence and structure. The fragment can include a C-terminal deletion an N-terminal deletion, and/or an internal deletion of the polypeptide. Active fragments of a particular protein or polypeptide will generally include at least about 5-14 contiguous amino acid residues of the full length molecule, but may include at least about 15-25 contiguous amino acid residues of the full length molecule, and can include at least about 20-50 or more contiguous amino acid residues of the full length molecule, or any integer between 5 amino acids and the full length sequence, provided that the fragment in question retains biological activity, such as inhibitory activity, as defined herein (e.g., the ability to inhibit MARK, or decrease tau or PSD-95 hyperphosphorylation, or decrease deposition of neurofibrillary tangles, or decrease loss of synaptic markers, dendritic spines, or synapses).

“Substantially purified” generally refers to isolation of a substance (compound, polynucleotide, protein, polypeptide, peptide composition) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

By “isolated” is meant, when referring to a polypeptide or peptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

A peptide is said to “interact” with a protein if it binds specifically (e.g., in a lock-and-key type mechanism), non-specifically or in some combination of specific and non-specific binding. A first peptide “interacts preferentially” with a protein if it binds (non-specifically and/or specifically) to the protein with greater affinity and/or greater specificity than it binds to other proteins (e.g., binds to MARK to a greater degree than to other proteins). The term “affinity” refers to the strength of binding and can be expressed quantitatively as a dissociation constant (Kd). It is to be understood that specific binding does not necessarily require interaction between specific amino acid residues and/or motifs of each peptide. For example, in certain embodiments, the peptides described herein interact preferentially with MARK but, nonetheless, may be capable of binding other polypeptides at a weak, yet detectable, level (e.g., 10% or less of the binding shown to the polypeptide of interest). Typically, weak binding, or background binding, is readily discernible from the preferential interaction with the compound or polypeptide of interest, e.g., by use of appropriate controls.

The term “MARK inhibitor” as used herein refers to any molecule (e.g., small molecule inhibitor, protein, peptide, nucleic acid, oligonucleotide, antibody, or fragment thereof) that inhibits MARK activity, and/or MARK expression, and/or the MARK signaling pathway, and/or tau phosphorylation, and/or PSD-95 phosphorylation. For example, the inhibitor may be a CagA protein, polypeptide, or peptide, or a nucleic acid encoding a CagA protein, polypeptide, or peptide. Inhibition may be complete or partial (i.e., all activity, some activity, or most activity is blocked by an inhibitor). For example, an inhibitor may reduce the activity of MARK by 10%, 20%, 30%, 40%, 50%, 60%, 70%, 80%, 90%, 100%, or any amount in between as compared to native or control levels.

The terms “label” and “detectable label” refer to a molecule capable of detection, including, but not limited to, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. The term “fluorescer” refers to a substance or a portion thereof that is capable of exhibiting fluorescence in the detectable range. Particular examples of labels that may be used with the invention include, but are not limited to radiolabels (e.g., ³H, ¹²⁵I, ³⁵S, ¹⁴C, or ³²P), phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin or other streptavidin-binding proteins, magnetic beads, electron dense reagents, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), Dronpa, Padron, mApple, mCherry, rsCherry, rsCherryRev, firefly luciferase, Renilla luciferase, NADPH, beta-galactosidase, horseradish peroxidase, glucose oxidase, alkaline phosphatase, chloramphenical acetyl transferase, and urease.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

An “effective amount” of a CagA peptide, polypeptide, or nucleic acid is an amount sufficient to effect beneficial or desired results, such as an amount that inhibits MARK, and/or reduces or prevents hyperphosphorylation of tau or PSD-95, and/or reduces or prevents deposition of neurofibrillary tangles, and/or reduces or prevents loss of expression of synaptic markers, and/or reduces or prevents loss of dendritic spines, and/or reduces or prevents loss of synapses. An effective amount can be administered in one or more administrations, applications or dosages.

By “therapeutically effective dose or amount” of a CagA peptide, polypeptide, or nucleic acid is intended an amount that, when administered as described herein, brings about a positive therapeutic response, such as improved neurological recovery from Alzheimer's disease or other tauopathy. Improved neurological recovery may include a reduction in deposition of neurofibrillary tangles or improved memory and cognitive function. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

By “subject” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like.

By “tauopathy” is meant a neurodegenerative disease associated with pathological aggregation of tau protein in the brain. Tauopathies include, but are not limited to, Alzheimer's disease, progressive supranuclear palsy, dementia pugilistica (chronic traumatic encephalopathy), frontotemporal dementia, frontotemporal lobar degeneration, parkinsonism linked to chromosome 17, lytico-Bodig disease (Parkinson-dementia complex of Guam), ganglioglioma, gangliocytoma, meningioangiomatosis, subacute sclerosing panencephalitis, lead encephalopathy, tuberous sclerosis, Hallervorden-Spatz disease, lipofuscinosis, Pick's disease, corticobasal degeneration, Argyrophilic grain disease (AGD), and tangle-predominant dementia, with NFTs similar to AD, but without plaques.

“Homology” refers to the percent identity between two polynucleotide or two polypeptide moieties. Two nucleic acid, or two polypeptide sequences are “substantially homologous” to each other when the sequences exhibit at least about 50% sequence identity, preferably at least about 75% sequence identity, more preferably at least about 80% 85% sequence identity, more preferably at least about 90% sequence identity, and most preferably at least about 95% 98% sequence identity over a defined length of the molecules. As used herein, substantially homologous also refers to sequences showing complete identity to the specified sequence.

In general, “identity” refers to an exact nucleotide to nucleotide or amino acid to amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353 358, National biomedical Research Foundation, Washington, D.C., which adapts the local homology algorithm of Smith and Waterman Advances in Appl. Math. 2:482 489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non redundant, GenBank+EMBL+DDBJ+PDB+GenBank CDS translations+Swiss protein+Spupdate+PIR. Details of these programs are readily available.

Alternatively, homology can be determined by hybridization of polynucleotides under conditions which form stable duplexes between homologous regions, followed by digestion with single stranded specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

“Recombinant” as used herein to describe a nucleic acid molecule means a polynucleotide of genomic, cDNA, viral, semisynthetic, or synthetic origin which, by virtue of its origin or manipulation, is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

The term “transformation” refers to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

“Recombinant host cells”, “host cells,” “cells”, “cell lines,” “cell cultures”, and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

“Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence.

“Expression cassette” or “expression construct” refers to an assembly which is capable of directing the expression of the sequence(s) or gene(s) of interest. An expression cassette generally includes control elements, as described above, such as a promoter which is operably linked to (so as to direct transcription of) the sequence(s) or gene(s) of interest, and often includes a polyadenylation sequence as well. Within certain embodiments of the invention, the expression cassette described herein may be contained within a plasmid construct. In addition to the components of the expression cassette, the plasmid construct may also include, one or more selectable markers, a signal which allows the plasmid construct to exist as single stranded DNA (e.g., a M13 origin of replication), at least one multiple cloning site, and a “mammalian” origin of replication (e.g., a SV40 or adenovirus origin of replication).

“Purified polynucleotide” refers to a polynucleotide of interest or fragment thereof which is essentially free, e.g., contains less than about 50%, preferably less than about 70%, and more preferably less than about at least 90%, of the protein with which the polynucleotide is naturally associated. Techniques for purifying polynucleotides of interest are well-known in the art and include, for example, disruption of the cell containing the polynucleotide with a chaotropic agent and separation of the polynucleotide(s) and proteins by ion-exchange chromatography, affinity chromatography and sedimentation according to density.

The term “transfection” is used to refer to the uptake of foreign DNA by a cell. A cell has been “transfected” when exogenous DNA has been introduced inside the cell membrane. A number of transfection techniques are generally known in the art. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA moieties into suitable host cells. The term refers to both stable and transient uptake of the genetic material, and includes uptake of peptide- or antibody-linked DNAs.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

The terms “variant,” “analog” and “mutein” refer to biologically active derivatives of the reference molecule that retain desired activity, such as the ability to inhibit MARK and reduce tau hyperphosphorylation. In general, the terms “variant” and “analog” refer to compounds having a native polypeptide sequence and structure with one or more amino acid additions, substitutions (generally conservative in nature) and/or deletions, relative to the native molecule, so long as the modifications do not destroy biological activity and which are “substantially homologous” to the reference molecule as defined below. In general, the amino acid sequences of such analogs will have a high degree of sequence homology to the reference sequence, e.g., amino acid sequence homology of more than 50%, generally more than 60%-70%, even more particularly 80%-85% or more, such as at least 90%-95% or more, when the two sequences are aligned. Often, the analogs will include the same number of amino acids but will include substitutions, as explained herein. The term “mutein” further includes polypeptides having one or more amino acid-like molecules including but not limited to compounds comprising only amino and/or imino molecules, polypeptides containing one or more analogs of an amino acid (including, for example, unnatural amino acids, etc.), polypeptides with substituted linkages, as well as other modifications known in the art, both naturally occurring and non-naturally occurring (e.g., synthetic), cyclized, branched molecules and the like. The term also includes molecules comprising one or more N-substituted glycine residues (a “peptoid”) and other synthetic amino acids or peptides. (See, e.g., U.S. Pat. Nos. 5,831,005; 5,877,278; and 5,977,301; Nguyen et al., Chem. Biol. (2000) 7:463-473; and Simon et al., Proc. Natl. Acad. Sci. USA (1992) 89:9367-9371 for descriptions of peptoids). Methods for making polypeptide analogs and muteins are known in the art and are described further below.

As explained above, analogs generally include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, and tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include up to about 5-10 conservative or non-conservative amino acid substitutions, or even up to about 15-25 conservative or non-conservative amino acid substitutions, or any integer between 5-25, so long as the desired function of the molecule remains intact. One of skill in the art may readily determine regions of the molecule of interest that can tolerate change by reference to Hopp/Woods and Kyte-Doolittle plots, well known in the art.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, alphaviruses, pox viruses and vaccinia viruses.

The term “derived from” is used herein to identify the original source of a molecule but is not meant to limit the method by which the molecule is made which can be, for example, by chemical synthesis or recombinant means.

A polynucleotide “derived from” a designated sequence refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

### II. Modes of Carrying Out the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on the discovery that peptide inhibitors of MARK can be used to block the toxic effects of amyloid-β on neurons, which lead to progression of Alzheimer's disease. In particular, the inventors have shown that exposure of rat hippocampal neurons to amyloid-β results in abnormal activation of MARK, which causes tau hyperphosphorylation, reduced expression of synaptic markers, and loss of dendritic spines and synapses. Treatment with a peptide inhibitor of MARK blocks these toxic effects of amyloid-β on neurons (see Example 1). These results indicate that peptide inhibitors of MARK will be useful as therapeutics for treating Alzheimer's disease and other tauopathies involving aberrant hyperphosphorylation of tau. In order to further an understanding of the invention, a more detailed discussion is provided below regarding methods of inhibiting MARK with peptide inhibitors and methods of using such peptide inhibitors for treating Alzheimer's disease and other disorders involving tau hyperphosphorylation.

A. MARK Inhibitors

In one aspect, the invention provides CagA peptides and polypeptides capable of binding, preferably specifically binding, to microtubule affinity regulating kinase (MARK) and inhibiting the activity of MARK. At least four isoforms of MARK exist, including MARK1, MARK2, MARK3, and MARK4. Thus, in certain embodiments one or more of the isoforms MARK1, MARK2, MARK3, and MARK4, or any combination thereof, are inhibited. Preferably, at least MARK4 is inhibited by the CagA peptide or polypeptide inhibitor. Inhibition may be complete or partial (i.e., all activity, some activity, or most activity is blocked by an inhibitor). For example, an inhibitor may reduce the activity of MARK by 10%, 20%, 30%, 40%, 50%, 60%, 70%, 80%, 90%, 100%, or any amount in between as compared to native or control levels.

The entire CagA protein or a polypeptide or peptide fragment thereof may be used to inhibit MARK. Biologically active fragments of CagA will generally include at least about 5-14 contiguous amino acid residues of the full length molecule, but may include at least about 15-25 contiguous amino acid residues of the full length molecule, and may include at least about 20-50 or more contiguous amino acid residues of the full length molecule, or any integer between 5 amino acids and the full length sequence, provided that the fragment in question retains biological activity, such as the ability to inhibit MARK. Additionally, MARK inhibitors derived from CagA may decrease tau or PSD-95 hyperphosphorylation, decrease deposition of neurofibrillary tangles, or decrease loss of synaptic markers, dendritic spines, or synapses.

CagA nucleic acid and protein sequences may be derived from Helicobacter pylori. A number of CagA nucleic acid and protein sequences are known. Representative CagA sequences are presented in SEQ ID NOS:1-5 and additional representative sequences are listed in the National Center for Biotechnology Information (NCBI) database. See, for example, NCBI entries: Accession Nos. AF247651, JQ318032, JQ318035, JQ318033, GU212666, GU212664, GU212662, GU212660, GU212658, GU212665, and GU212663; all of which sequences (as entered by the date of filing of this application) are herein incorporated by reference. Any of these sequences or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, can be used to construct a CagA peptide, polypeptide, or nucleic acid as described herein. If desired, CagA peptides or polypeptides can contain other amino acid sequences, such as amino acid linkers or signal sequences, as well as ligands or tags useful in protein purification.

In certain embodiments, tag sequences are located at the N-terminus or C-terminus of the CagA peptide or polypeptide. Exemplary tags that can be used in the practice of the invention include a His-tag, a Strep-tag, a TAP-tag, an S-tag, an SBP-tag, an Arg-tag, a calmodulin-binding peptide tag, a cellulose-binding domain tag, a DsbA tag, a c-myc tag, a glutathione S-transferase tag, a FLAG tag, a HAT-tag, a maltose-binding protein tag, a NusA tag, and a thioredoxin tag.

In certain embodiments, the CagA peptide or polypeptide further comprises one or more linkers. Linkers are typically short peptide sequences of 2-30 amino acid residues, often composed of glycine and/or serine residues. Linker amino acid sequences will typically be short, e.g., 20 or fewer amino acids (i.e., 20, 19, 18, 17, 16, 15, 14, 13, 12, 11, 10, 9, 8, 7, 6, 5, 4, 3, 2, or 1). Examples include short peptide sequences which facilitate cloning, poly-glycine linkers (Glyₙ where n=2, 3, 4, 5, 6, 7, 8, 9, 10 or more), histidine tags (Hisₙ where n=3, 4, 5, 6, 7, 8, 9, 10 or more), linkers composed of glycine and serine residues ([Gly-Ser]ₙ, [Gly-Gly-Ser-Gly]ₙ (SEQ ID NO:6), [Ser-Ala-Gly-Gly]ₙ (SEQ ID NO:7), and [Gly-Gly-Gly-Gly-Ser]ₙ (SEQ ID NO:8), wherein n=1, 2, 3, 4, 5, 6, 7, 8, 9, 10, 11, 12, 13, 14, 15 or more), GSAT, SEG, and Z-EGFR linkers. Linkers may include restriction sites, which aid cloning and manipulation. Other suitable linker amino acid sequences will be apparent to those skilled in the art. (See e.g., Argos (1990) J. Mol. Biol. 211(4):943-958; Crasto et al. (2000) Protein Eng. 13:309-312; George et al. (2002) Protein Eng. 15:871-879; Arai et al. (2001) Protein Eng. 14:529-532; and the Registry of Standard Biological Parts (partsregistry.org/Protein_domains/Linker).

In certain embodiments, the CagA peptide or polypeptide may comprise a detectable label in order to facilitate detection of binding of the peptide or polypeptide to MARK. Detectable labels suitable for use in the present invention include any composition detectable by spectroscopic, photochemical, biochemical, immunochemical, electrical, optical, or chemical means. Particular examples of labels that may be used with the invention include, but are not limited to radiolabels (e.g., ³H, ¹²⁵I, ³⁵S, ¹⁴C, or ³²P), phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), Dronpa, Padron, mApple, mCherry, rsCherry, rsCherryRev, firefly luciferase, Renilla luciferase, biotin or other streptavidin-binding proteins, magnetic beads, electron dense reagents, NADPH, beta-galactosidase, horseradish peroxidase, glucose oxidase, alkaline phosphatase, chloramphenical acetyl transferase, and urease. Patents teaching the use of such labels include U.S. Pat. Nos. 3,817,837; 3,850,752; 3,939,350; 3,996,345; 4,277,437; 4,275,149; and 4,366,241.

In certain embodiments, the CagA peptide or polypeptide is linked to an internalization sequence, a protein transduction domain, or a cell penetrating peptide to facilitate entry into a cell. Cell penetrating peptides that can be used in the practice of the invention include, but are not limited to, HIV-Tat, penetratin, transportan, octaarginine, nonaarginine, antennapedia, TP10, Buforin II, MAP (model amphipathic peptide), K-FGF, Ku70, mellittin, pVEC, Pep-1, SynB1, Pep-7, CADY, GALA, pHLIP, KALA, R7W, and HN-1, which can readily transport molecules and small peptides across plasma membranes (see, e.g., Jones et al. (2012) “Cell entry of cell penetrating peptides and tales of tails wagging dogs,” J. Control Release. 2012, in press; Fonseca et al. (2009) Adv. Drug Deliv. Rev. 61(11):953-64; Schwarze et al. (1999) Science. 285(5433):1569-72; Derossi et al. (1996) J. Biol. Chem. 271(30):18188-18193; Fuchs et al. (2004) Biochemistry 43(9):2438-2444; and Yuan et al. (2002) Cancer Res. 62(15):4186-4190).

In certain embodiments, the MARK inhibitor is selected from the group consisting of:
a) a peptide comprising the amino acid sequence of SEQ ID NO: 1;
b) a peptide comprising an amino acid sequence having at least 95% identity to the sequence of SEQ ID NO: 1, wherein the peptide inhibits the activity of MARK;
c) a polypeptide comprising the amino acid sequence of SEQ ID NO: 2;
d) a polypeptide comprising an amino acid sequence having at least 95% identity to the sequence of SEQ ID NO: 2, wherein the polypeptide inhibits the activity of MARK;
e) a polypeptide comprising the amino acid sequence of SEQ ID NO: 4;
f) a polypeptide comprising an amino acid sequence having at least 95% identity to the sequence of SEQ ID NO: 4, wherein the polypeptide inhibits the activity of MARK; and
g) a CagA peptide of 14 to 50 amino acids in length comprising at least residues 941 to 954 of the amino acid sequence of SEQ ID NO:4.

In other embodiments, the MARK inhibitor is a recombinant polynucleotide comprising a promoter operably linked to a polynucleotide encoding an inhibitor of MARK (e.g., a CagA protein, polypeptide, or peptide). The recombinant polynucleotide may comprise an expression vector, for example, a bacterial plasmid vector or a viral expression vector (e.g., an adenovirus, retrovirus (e.g., γ-retrovirus and lentivirus), poxvirus, adeno-associated virus, baculovirus, or herpes simplex virus vector).

In certain embodiments, the recombinant polynucleotide expressing the inhibitor of MARK, comprises a polynucleotide selected from the group consisting of:
a) a polynucleotide encoding a peptide comprising the amino acid sequence of SEQ ID NO: 1;
b) a polynucleotide encoding a peptide comprising an amino acid sequence having at least 95% identity to the sequence of SEQ ID NO: 1, wherein the peptide inhibits the activity of MARK;
c) a polynucleotide encoding a polypeptide comprising the amino acid sequence of SEQ ID NO: 2;
d) a polynucleotide encoding a polypeptide comprising an amino acid sequence having at least 95% identity to the sequence of SEQ ID NO: 2, wherein the polypeptide inhibits the activity of MARK;
e) a polynucleotide encoding a polypeptide comprising the amino acid sequence of SEQ ID NO: 4;
f) a polynucleotide encoding a polypeptide comprising an amino acid sequence having at least 95% identity to the sequence of SEQ ID NO: 4, wherein the polypeptide inhibits the activity of MARK;
g) a polynucleotide encoding a CagA peptide of 14 to 50 amino acids in length, said peptide comprising at least residues 941 to 954 of the amino acid sequence of SEQ ID NO:4;
h) a CagA polynucleotide of 42 to 150 nucleotides in length comprising at least nucleotides 2821 to 2862 of the nucleotide sequence of SEQ ID NO:3;
i) a polynucleotide comprising the nucleotide sequence of SEQ ID NO:5; and
j) a polynucleotide comprising a nucleotide sequence having at least 95% identity to the sequence of SEQ ID NO:5, wherein the polynucleotide encodes a polypeptide that inhibits the activity of MARK.

B. Production of CagA Peptides and Polypeptides

CagA polypeptides and peptide fragments thereof can be prepared in any suitable manner (e.g., recombinant expression, purification from cell culture, chemical synthesis, etc.) and in various forms (e.g. native, fusions, labeled, lipidated, etc.). CagA polypeptides include naturally-occurring polypeptides, recombinantly produced polypeptides, synthetically produced polypeptides, or polypeptides produced by a combination of these methods. Means for preparing peptides and polypeptides are well understood in the art. Peptides and polypeptides are preferably prepared in substantially pure form (i.e. substantially free from other host cell or non-host cell proteins).

In one embodiment, the peptides or polypeptides are generated using recombinant techniques. One of skill in the art can readily determine nucleotide sequences that encode the desired peptides or polypeptides using standard methodology and the teachings herein. Oligonucleotide probes can be devised based on the known sequences and used to probe genomic or cDNA libraries. The sequences can then be further isolated using standard techniques and, e.g., restriction enzymes employed to truncate the gene at desired portions of the full-length sequence. Similarly, sequences of interest can be isolated directly from cells and tissues containing the same, using known techniques, such as phenol extraction and the sequence further manipulated to produce the desired truncations. See, e.g., Sambrook et al., supra, for a description of techniques used to obtain and isolate DNA.

The sequences encoding peptides or polypeptides can also be produced synthetically, for example, based on the known sequences. The nucleotide sequence can be designed with the appropriate codons for the particular amino acid sequence desired. The complete sequence is generally assembled from overlapping oligonucleotides prepared by standard methods and assembled into a complete coding sequence. See, e.g., Edge (1981) Nature 292:756; Nambair et al. (1984) Science 223:1299; Jay et al. (1984) J. Biol. Chem. 259:6311; Stemmer et al. (1995) Gene 164:49-53.

Recombinant techniques are readily used to clone sequences encoding peptides or polypeptides that can then be mutagenized in vitro by the replacement of the appropriate base pair(s) to result in the codon for the desired amino acid. Such a change can include as little as one base pair, effecting a change in a single amino acid, or can encompass several base pair changes. Alternatively, the mutations can be effected using a mismatched primer that hybridizes to the parent nucleotide sequence (generally cDNA corresponding to the RNA sequence), at a temperature below the melting temperature of the mismatched duplex. The primer can be made specific by keeping primer length and base composition within relatively narrow limits and by keeping the mutant base centrally located. See, e.g., Innis et al, (1990) PCR Applications: Protocols for Functional Genomics; Zoller and Smith, Methods Enzymol. (1983) 100:468. Primer extension is effected using DNA polymerase, the product cloned and clones containing the mutated DNA, derived by segregation of the primer extended strand, selected. Selection can be accomplished using the mutant primer as a hybridization probe. The technique is also applicable for generating multiple point mutations. See, e.g., Dalbie-McFarland et al. Proc. Natl. Acad. Sci USA (1982) 79:6409.

Once coding sequences have been isolated and/or synthesized, they can be cloned into any suitable vector or replicon for expression. (See, also, Examples). As will be apparent from the teachings herein, a wide variety of vectors encoding modified peptides can be generated by creating expression constructs which operably link, in various combinations, polynucleotides encoding peptides having deletions or mutations therein.

Numerous cloning vectors are known to those of skill in the art, and the selection of an appropriate cloning vector is a matter of choice. Examples of recombinant DNA vectors for cloning and host cells which they can transform include the bacteriophage λ (E. coli), pBR322 (E. coli), pACYC177 (E. coli), pKT230 (gram-negative bacteria), pGV1106 (gram-negative bacteria), pLAFR1 (gram-negative bacteria), pME290 (non-E. coli gram-negative bacteria), pHV14 (E. coli and Bacillus subtilis), pBD9 (Bacillus), pIJ61 (Streptomyces), pUC6 (Streptomyces), YIp5 (Saccharomyces), YCp19 (Saccharomyces) and bovine papilloma virus (mammalian cells). See, generally, DNA Cloning: Vols. I & II, supra; Sambrook et al., supra; B. Perbal, supra.

Insect cell expression systems, such as baculovirus systems, can also be used and are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif. (“MaxBac” kit).

Plant expression systems can also be used to produce the CagA peptides and polypeptides described herein. Generally, such systems use virus-based vectors to transfect plant cells with heterologous genes. For a description of such systems, see, e.g., Porta et al., Mol. Biotech. (1996) 5:209-221; and Hackland et al., Arch. Virol. (1994) 139:1-22.

Viral systems, such as a vaccinia based infection/transfection system, as described in Tomei et al., J. Virol. (1993) 67:4017-4026 and Selby et al., J. Gen. Virol. (1993) 74:1103-1113, will also find use with the present invention. In this system, cells are first transfected in vitro with a vaccinia virus recombinant that encodes the bacteriophage T7 RNA polymerase. This polymerase displays exquisite specificity in that it only transcribes templates bearing T7 promoters. Following infection, cells are transfected with the DNA of interest, driven by a T7 promoter. The polymerase expressed in the cytoplasm from the vaccinia virus recombinant transcribes the transfected DNA into RNA that is then translated into protein by the host translational machinery. The method provides for high level, transient, cytoplasmic production of large quantities of RNA and its translation product(s).

The gene can be placed under the control of a promoter, ribosome binding site (for bacterial expression) and, optionally, an operator (collectively referred to herein as “control” elements), so that the DNA sequence encoding the desired polypeptide is transcribed into RNA in the host cell transformed by a vector containing this expression construction. The coding sequence may or may not contain a signal peptide or leader sequence. With the present invention, both the naturally occurring signal peptides or heterologous sequences can be used. Leader sequences can be removed by the host in post-translational processing. See, e.g., U.S. Pat. Nos. 4,431,739; 4,425,437; 4,338,397. Such sequences include, but are not limited to, the TPA leader, as well as the honey bee mellitin signal sequence.

Other regulatory sequences may also be desirable which allow for regulation of expression of the protein sequences relative to the growth of the host cell. Such regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

The control sequences and other regulatory sequences may be ligated to the coding sequence prior to insertion into a vector. Alternatively, the coding sequence can be cloned directly into an expression vector that already contains the control sequences and an appropriate restriction site.

In some cases it may be necessary to modify the coding sequence so that it may be attached to the control sequences with the appropriate orientation; i.e., to maintain the proper reading frame. Mutants or analogs may be prepared by the deletion of a portion of the sequence encoding the protein, by insertion of a sequence, and/or by substitution of one or more nucleotides within the sequence. Techniques for modifying nucleotide sequences, such as site-directed mutagenesis, are well known to those skilled in the art. See, e.g., Sambrook et al., supra; DNA Cloning, Vols. I and II, supra; Nucleic Acid Hybridization, supra.

The expression vector is then used to transform an appropriate host cell. A number of mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human hepatocellular carcinoma cells (e.g., Hep G2), Vero293 cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorphs, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frugiperda, and Trichoplusia ni.

Depending on the expression system and host selected, the fusion proteins of the present invention are produced by growing host cells transformed by an expression vector described above under conditions whereby the protein of interest is expressed. The selection of the appropriate growth conditions is within the skill of the art.

In one embodiment, the transformed cells secrete the peptide or polypeptide product into the surrounding media. Certain regulatory sequences can be included in the vector to enhance secretion of the protein product, for example using a tissue plasminogen activator (TPA) leader sequence, an interferon (γ or α) signal sequence or other signal peptide sequences from known secretory proteins. The secreted peptide or polypeptide product can then be isolated by various techniques described herein, for example, using standard purification techniques such as but not limited to, hydroxyapatite resins, column chromatography, ion-exchange chromatography, size-exclusion chromatography, electrophoresis, HPLC, immunoadsorbent techniques, affinity chromatography, immunoprecipitation, and the like.

Alternatively, the transformed cells are disrupted, using chemical, physical or mechanical means, which lyse the cells yet keep the recombinant peptides or polypeptides substantially intact. Intracellular proteins can also be obtained by removing components from the cell wall or membrane, e.g., by the use of detergents or organic solvents, such that leakage of the polypeptides occurs. Such methods are known to those of skill in the art and are described in, e.g., Protein Purification Applications: A Practical Approach, (Simon Roe, Ed., 2001).

For example, methods of disrupting cells for use with the present invention include but are not limited to: sonication or ultrasonication; agitation; liquid or solid extrusion; heat treatment; freeze-thaw; desiccation; explosive decompression; osmotic shock; treatment with lytic enzymes including proteases such as trypsin, neuraminidase and lysozyme; alkali treatment; and the use of detergents and solvents such as bile salts, sodium dodecylsulphate, Triton, NP40 and CHAPS. The particular technique used to disrupt the cells is largely a matter of choice and will depend on the cell type in which the polypeptide is expressed, culture conditions and any pre-treatment used.

Following disruption of the cells, cellular debris is removed, generally by centrifugation, and the intracellularly produced peptides or polypeptides are further purified, using standard purification techniques such as but not limited to, column chromatography, ion-exchange chromatography, size-exclusion chromatography, electrophoresis, HPLC, immunoadsorbent techniques, affinity chromatography, immunoprecipitation, and the like.

For example, one method for obtaining the intracellular peptides or polypeptides of the present invention involves affinity purification, such as by immunoaffinity chromatography using antibodies (e.g., previously generated antibodies), or by lectin affinity chromatography. Particularly preferred lectin resins are those that recognize mannose moieties such as but not limited to resins derived from Galanthus nivalis agglutinin (GNA), Lens culinaris agglutinin (LCA or lentil lectin), Pisum sativum agglutinin (PSA or pea lectin), Narcissus pseudonarcissus agglutinin (NPA) and Allium ursinum agglutinin (AUA). The choice of a suitable affinity resin is within the skill in the art. After affinity purification, the peptides or polypeptides can be further purified using conventional techniques well known in the art, such as by any of the techniques described above.

CagA peptides and polypeptides can be conveniently synthesized chemically, for example by any of several techniques that are known to those skilled in the peptide art. See, e.g., Fmoc Solid Phase Peptide Synthesis: A Practical Approach (W. C. Chan and Peter D. White eds., Oxford University Press, 1^{st} edition, 2000); N. Leo Benoiton, Chemistry of Peptide Synthesis (CRC Press; 1^{st} edition, 2005); Peptide Synthesis and Applications (Methods in Molecular Biology, John Howl ed., Humana Press, 1^{st} ed., 2005); and Pharmaceutical Formulation Development of Peptides and Proteins (The Taylor & Francis Series in Pharmaceutical Sciences, Lars Hovgaard, Sven Frokjaer, and Marco van de Weert eds., CRC Press; 1^{st} edition, 1999); herein incorporated by reference.

In general, these methods employ the sequential addition of one or more amino acids to a growing peptide chain. Normally, either the amino or carboxyl group of the first amino acid is protected by a suitable protecting group. The protected or derivatized amino acid can then be either attached to an inert solid support or utilized in solution by adding the next amino acid in the sequence having the complementary (amino or carboxyl) group suitably protected, under conditions that allow for the formation of an amide linkage. The protecting group is then removed from the newly added amino acid residue and the next amino acid (suitably protected) is then added, and so forth. After the desired amino acids have been linked in the proper sequence, any remaining protecting groups (and any solid support, if solid phase synthesis techniques are used) are removed sequentially or concurrently, to render the final peptide or polypeptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide. See, e.g., J. M. Stewart and J. D. Young, Solid Phase Peptide Synthesis (Pierce Chemical Co., Rockford, Ill. 1984) and G. Barany and R. B. Merrifield, The Peptides: Analysis, Synthesis, Biology, editors E. Gross and J. Meienhofer, Vol. 2, (Academic Press, New York, 1980), pp. 3-254, for solid phase peptide synthesis techniques; and M. Bodansky, Principles of Peptide Synthesis, (Springer-Verlag, Berlin 1984) and E. Gross and J. Meienhofer, Eds., The Peptides: Analysis, Synthesis, Biology, Vol. 1, for classical solution synthesis. These methods are typically used for relatively small polypeptides, i.e., up to about 50-100 amino acids in length, but are also applicable to larger polypeptides.

Typical protecting groups include t-butyloxycarbonyl (Boc), 9-fluorenylmethoxycarbonyl (Fmoc) benzyloxycarbonyl (Cbz); p-toluenesulfonyl (Tx); 2,4-dinitrophenyl; benzyl (Bzl); biphenylisopropyloxycarboxy-carbonyl, t-amyloxycarbonyl, isobornyloxycarbonyl, o-bromobenzyloxycarbonyl, cyclohexyl, isopropyl, acetyl, o-nitrophenylsulfonyl and the like.

Typical solid supports are cross-linked polymeric supports. These can include divinylbenzene cross-linked-styrene-based polymers, for example, divinylbenzene-hydroxymethylstyrene copolymers, divinylbenzene-chloromethylstyrene copolymers and divinylbenzene-benzhydrylaminopolystyrene copolymers.

CagA peptides and polypeptides can also be chemically prepared by other methods such as by the method of simultaneous multiple peptide synthesis. See, e.g., Houghten Proc. Natl. Acad. Sci. USA (1985) 82:5131-5135; U.S. Pat. No. 4,631,211.

C. Nucleic Acids Encoding CagA Peptides and Polypeptides

Nucleic acids encoding MARK inhibitors, such as CagA peptides or polypeptides can be used, for example, to treat Alzheimer's disease and other tauopathies. Nucleic acids described herein can be inserted into an expression vector to create an expression cassette capable of producing the CagA peptides or polypeptides in a suitable host cell. The ability of constructs to produce the CagA peptides and polypeptides can be empirically determined (e.g., see Example 1 describing detection of a GFP-tagged CagA peptide by fluorescence microscopy).

Expression cassettes typically include control elements operably linked to the coding sequence, which allow for the expression of the gene in vivo in the subject species. For example, typical promoters for mammalian cell expression include the SV40 early promoter, a CMV promoter such as the CMV immediate early promoter, the mouse mammary tumor virus LTR promoter, the adenovirus major late promoter (Ad MLP), and the herpes simplex virus promoter, among others. Other nonviral promoters, such as a promoter derived from the murine metallothionein gene, will also find use for mammalian expression. Typically, transcription termination and polyadenylation sequences will also be present, located 3′ to the translation stop codon. Preferably, a sequence for optimization of initiation of translation, located 5′ to the coding sequence, is also present. Examples of transcription terminator/polyadenylation signals include those derived from SV40, as described in Sambrook et al., supra, as well as a bovine growth hormone terminator sequence.

Enhancer elements may also be used herein to increase expression levels of the mammalian constructs. Examples include the SV40 early gene enhancer, as described in Dijkema et al., EMPO J. (1985) 4:761, the enhancer/promoter derived from the long terminal repeat (LTR) of the Rous Sarcoma Virus, as described in Gorman et al., Proc. Natl. Acad. Sci. USA (1982b) 79:6777 and elements derived from human CMV, as described in Boshart et al., Cell (1985) 41:521, such as elements included in the CMV intron A sequence.

Once complete, the constructs encoding CagA peptides or polypeptides can be administered to a subject using standard gene delivery protocols. Methods for gene delivery are known in the art. See, e.g., U.S. Pat. Nos. 5,399,346, 5,580,859, 5,589,466. Genes can be delivered either directly to a vertebrate subject or, alternatively, delivered ex vivo, to cells derived from the subject and the cells reimplanted in the subject.

A number of viral based systems have been developed for gene transfer into mammalian cells. These include adenoviruses, retroviruses (γ-retroviruses and lentiviruses), poxviruses, adeno-associated viruses, baculoviruses, and herpes simplex viruses (see e.g., Warnock et al. (2011) Methods Mol. Biol. 737:1-25; Walther et al. (2000) Drugs 60(2):249-271; and Lundstrom (2003) Trends Biotechnol. 21(3):117-122; herein incorporated by reference).

For example, retroviruses provide a convenient platform for gene delivery systems. Selected sequences can be inserted into a vector and packaged in retroviral particles using techniques known in the art. The recombinant virus can then be isolated and delivered to cells of the subject either in vivo or ex vivo. A number of retroviral systems have been described (U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; Boris-Lawrie and Temin (1993) Cur. Opin. Genet. Develop. 3:102-109; and Ferry et al. (2011) Curr Pharm Des. 17(24):2516-2527). Lentiviruses are a class of retroviruses that are particularly useful for delivering polynucleotides to mammalian cells because they are able to infect both dividing and nondividing cells (see e.g., Lois et al (2002) Science 295:868-872; Durand et al. (2011) Viruses 3(2):132-159; herein incorporated by reference).

A number of adenovirus vectors have also been described. Unlike retroviruses which integrate into the host genome, adenoviruses persist extrachromosomally thus minimizing the risks associated with insertional mutagenesis (Haj-Ahmad and Graham, J. Virol. (1986) 57:267-274; Bett et al., J. Virol. (1993) 67:5911-5921; Mittereder et al., Human Gene Therapy (1994) 5:717-729; Seth et al., J. Virol. (1994) 68:933-940; Barr et al., Gene Therapy (1994) 1:51-58; Berkner, K. L. BioTechniques (1988) 6:616-629; and Rich et al., Human Gene Therapy (1993) 4:461-476). Additionally, various adeno-associated virus (AAV) vector systems have been developed for gene delivery. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al., Molec. Cell. Biol. (1988) 8:3988-3996; Vincent et al., Vaccines 90 (1990) (Cold Spring Harbor Laboratory Press); Carter, B. J. Current Opinion in Biotechnology (1992) 3:533-539; Muzyczka, N. Current Topics in Microbiol. and Immunol. (1992) 158:97-129; Kotin, R. M. Human Gene Therapy (1994) 5:793-801; Shelling and Smith, Gene Therapy (1994) 1:165-169; and Zhou et al., J. Exp. Med. (1994) 179:1867-1875.

Another vector system useful for delivering the polynucleotides of the present invention is the enterically administered recombinant poxvirus vaccines described by Small, Jr., P. A., et al. (U.S. Pat. No. 5,676,950, issued Oct. 14, 1997, herein incorporated by reference).

Additional viral vectors which will find use for delivering the nucleic acid molecules encoding the CagA peptides and polypeptides of interest include those derived from the pox family of viruses, including vaccinia virus and avian poxvirus. By way of example, vaccinia virus recombinants expressing the CagA peptides or polypeptides can be constructed as follows. The DNA encoding the particular CagA peptide or polypeptide coding sequence is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the coding sequences of interest into the viral genome. The resulting TK-recombinant can be selected by culturing the cells in the presence of 5-bromodeoxyuridine and picking viral plaques resistant thereto.

Alternatively, avipoxviruses, such as the fowlpox and canarypox viruses, can also be used to deliver the genes. Recombinant avipox viruses, expressing immunogens from mammalian pathogens, are known to confer protective immunity when administered to non-avian species. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with. respect to the production of vaccinia viruses. See, e.g., WO 91/12882; WO 89/03429; and WO 92/03545.

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the Alphavirus genus, such as, but not limited to, vectors derived from the Sindbis virus (SIN), Semliki Forest virus (SFV), and Venezuelan Equine Encephalitis virus (VEE), will also find use as viral vectors for delivering the polynucleotides of the present invention. For a description of Sindbis-virus derived vectors useful for the practice of the instant methods, see, Dubensky et al. (1996) J. Virol. 70:508-519; and International Publication Nos. WO 95/07995, WO 96/17072; as well as, Dubensky, Jr., T. W., et al., U.S. Pat. No. 5,843,723, issued Dec. 1, 1998, and Dubensky, Jr., T. W., U.S. Pat. No. 5,789,245, issued Aug. 4, 1998, both herein incorporated by reference. Particularly preferred are chimeric alphavirus vectors comprised of sequences derived from Sindbis virus and Venezuelan equine encephalitis virus. See, e.g., Perri et al. (2003) J. Viol. 77: 10394-10403 and International Publication Nos. WO 02/099035, WO 02/080982, WO 01/81609, and WO 00/61772; herein incorporated by reference in their entireties.

A vaccinia based infection/transfection system can be conveniently used to provide for inducible, transient expression of the coding sequences of interest (for example, a CagA peptide expression cassette) in a host cell. In this system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide of interest, driven by a T7 promoter. The polymerase expressed in the cytoplasm from the vaccinia virus recombinant transcribes the transfected DNA into RNA which is then translated into protein by the host translational machinery. The method provides for high level, transient, cytoplasmic production of large quantities of RNA and its translation products. See, e.g., Elroy-Stein and Moss, Proc. Natl. Acad. Sci. USA (1990) 87:6743-6747; Fuerst et al., Proc. Natl. Acad. Sci. USA (1986) 83:8122-8126.

As an alternative approach to infection with vaccinia or avipox virus recombinants, or to the delivery of genes using other viral vectors, an amplification system can be used that will lead to high level expression following introduction into host cells. Specifically, a T7 RNA polymerase promoter preceding the coding region for T7 RNA polymerase can be engineered. Translation of RNA derived from this template will generate T7 RNA polymerase which in turn will transcribe more template. Concomitantly, there will be a cDNA whose expression is under the control of the T7 promoter. Thus, some of the T7 RNA polymerase generated from translation of the amplification template RNA will lead to transcription of the desired gene. Because some T7 RNA polymerase is required to initiate the amplification, T7 RNA polymerase can be introduced into cells along with the template(s) to prime the transcription reaction. The polymerase can be introduced as a protein or on a plasmid encoding the RNA polymerase. For a further discussion of T7 systems and their use for transforming cells, see, e.g., International Publication No. WO 94/26911; Studier and Moffatt, J. Mol. Biol. (1986) 189:113-130; Deng and Wolff, Gene (1994) 143:245-249; Gao et al., Biochem. Biophys. Res. Commun. (1994) 200:1201-1206; Gao and Huang, Nuc. Acids Res. (1993) 21:2867-2872; Chen et al., Nuc. Acids Res. (1994) 22:2114-2120; and U.S. Pat. No. 5,135,855.

The synthetic expression cassette of interest can also be delivered without a viral vector. For example, the synthetic expression cassette can be packaged as DNA or RNA in liposomes prior to delivery to the subject or to cells derived therefrom. Lipid encapsulation is generally accomplished using liposomes which are able to stably bind or entrap and retain nucleic acid. The ratio of condensed DNA to lipid preparation can vary but will generally be around 1:1 (mg DNA:micromoles lipid), or more of lipid. For a review of the use of liposomes as carriers for delivery of nucleic acids, see, Hug and Sleight, Biochim. Biophys. Acta. (1991) 1097:1-17; Straubinger et al., in Methods of Enzymology (1983), Vol. 101, pp. 512-527.

Liposomal preparations for use in the present invention include cationic (positively charged), anionic (negatively charged) and neutral preparations, with cationic liposomes particularly preferred. Cationic liposomes have been shown to mediate intracellular delivery of plasmid DNA (Felgner et al., Proc. Natl. Acad. Sci. USA (1987) 84:7413-7416); mRNA (Malone et al., Proc. Natl. Acad. Sci. USA (1989) 86:6077-6081); and purified transcription factors (Debs et al., J. Biol. Chem. (1990) 265:10189-10192), in functional form.

Cationic liposomes are readily available. For example, N[1-2,3-dioleyloxy)propyl]-N,N,N-triethylammonium (DOTMA) liposomes are available under the trademark Lipofectin, from GIBCO BRL, Grand Island, N.Y. (See, also, Felgner et al., Proc. Natl. Acad. Sci. USA (1987) 84:7413-7416). Other commercially available lipids include (DDAB/DOPE) and DOTAP/DOPE (Boerhinger). Other cationic liposomes can be prepared from readily available materials using techniques well known in the art. See, e.g., Szoka et al., Proc. Natl. Acad. Sci. USA (1978) 75:4194-4198; PCT Publication No. WO 90/11092 for a description of the synthesis of DOTAP (1,2-bis(oleoyloxy)-3-(trimethylammonio)propane) liposomes.

Similarly, anionic and neutral liposomes are readily available, such as, from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

The liposomes can comprise multilammelar vesicles (MLVs), small unilamellar vesicles (SUVs), or large unilamellar vesicles (LUVs). The various liposome-nucleic acid complexes are prepared using methods known in the art. See, e.g., Straubinger et al., in METHODS OF IMMUNOLOGY (1983), Vol. 101, pp. 512-527; Szoka et al., Proc. Natl. Acad. Sci. USA (1978) 75:4194-4198; Papahadjopoulos et al., Biochim. Biophys. Acta (1975) 394:483; Wilson et al., Cell (1979) 17:77); Deamer and Bangham, Biochim. Biophys. Acta (1976) 443:629; Ostro et al., Biochem. Biophys. Res. Commun. (1977) 76:836; Fraley et al., Proc. Natl. Acad. Sci. USA (1979) 76:3348); Enoch and Strittmatter, Proc. Natl. Acad. Sci. USA (1979) 76:145); Fraley et al., J. Biol. Chem. (1980) 255:10431; Szoka and Papahadjopoulos, Proc. Natl. Acad. Sci. USA (1978) 75:145; and Schaefer-Ridder et al., Science (1982) 215:166.

The DNA and/or peptide(s) can also be delivered in cochleate lipid compositions similar to those described by Papahadjopoulos et al., Biochem. Biophys. Acta. (1975) 394:483-491. See, also, U.S. Pat. Nos. 4,663,161 and 4,871,488.

The expression cassette of interest may also be encapsulated, adsorbed to, or associated with, particulate carriers. Examples of particulate carriers include those derived from polymethyl methacrylate polymers, as well as microparticles derived from poly(lactides) and poly(lactide-co-glycolides), known as PLG. See, e.g., Jeffery et al., Pharm. Res. (1993) 10:362-368; McGee J. P., et al., J Microencapsul. 14(2):197-210, 1997; O'Hagan D. T., et al., Vaccine 11(2):149-54, 1993.

Furthermore, other particulate systems and polymers can be used for the in vivo or ex vivo delivery of the nucleic acid of interest. For example, polymers such as polylysine, polyarginine, polyornithine, spermine, spermidine, as well as conjugates of these molecules, are useful for transferring a nucleic acid of interest. Similarly, DEAF dextran-mediated transfection, calcium phosphate precipitation or precipitation using other insoluble inorganic salts, such as strontium phosphate, aluminum silicates including bentonite and kaolin, chromic oxide, magnesium silicate, talc, and the like, will find use with the present methods. See, e.g., Felgner, P. L., Advanced Drug Delivery Reviews (1990) 5:163-187, for a review of delivery systems useful for gene transfer. Peptoids (Zuckerman, R. N., et al., U.S. Pat. No. 5,831,005, issued Nov. 3, 1998, herein incorporated by reference) may also be used for delivery of a construct of the present invention.

Additionally, biolistic delivery systems employing particulate carriers such as gold and tungsten, are especially useful for delivering synthetic expression cassettes of the present invention. The particles are coated with the synthetic expression cassette(s) to be delivered and accelerated to high velocity, generally under a reduced atmosphere, using a gun powder discharge from a “gene gun.” For a description of such techniques, and apparatuses useful therefore, see, e.g., U.S. Pat. Nos. 4,945,050; 5,036,006; 5,100,792; 5,179,022; 5,371,015; and 5,478,744. Also, needle-less injection systems can be used (Davis, H. L., et al, Vaccine 12:1503-1509, 1994; Bioject, Inc., Portland, Oreg.).

Recombinant vectors carrying a synthetic expression cassette of the present invention are formulated into compositions for delivery to a vertebrate subject. These compositions may either be prophylactic (to prevent tau hyperphosphorylation and formation of neurofibrillary tangles) or therapeutic (to treat Alzheimer's disease and other tauopathies). The compositions will comprise a “therapeutically effective amount” of the nucleic acid of interest such that an amount of the CagA peptide or polypeptide can be produced in vivo so that MARK is inhibited in the individual to which it is administered. The exact amount necessary will vary depending on the subject being treated; the age and general condition of the subject to be treated; the degree of protection desired; the severity of the condition being treated; the particular CagA peptide or polypeptide produced and its mode of administration, among other factors. An appropriate effective amount can be readily determined by one of skill in the art. Thus, a “therapeutically effective amount” will fall in a relatively broad range that can be determined through routine trials.

The compositions will generally include one or more “pharmaceutically acceptable excipients or vehicles” such as water, saline, glycerol, polyethyleneglycol, hyaluronic acid, ethanol, etc. Additionally, auxiliary substances, such as wetting or emulsifying agents, pH buffering substances, surfactants and the like, may be present in such vehicles. Certain facilitators of nucleic acid uptake and/or expression can also be included in the compositions or coadministered.

Once formulated, the compositions of the invention can be administered directly to the subject (e.g., as described above) or, alternatively, delivered ex vivo, to cells derived from the subject, using methods such as those described above. For example, methods for the ex vivo delivery and reimplantation of transformed cells into a subject are known in the art and can include, e.g., dextran-mediated transfection, calcium phosphate precipitation, polybrene mediated transfection, lipofectamine and LT-1 mediated transfection, protoplast fusion, electroporation, encapsulation of the polynucleotide(s) in liposomes, and direct microinjection of the DNA into nuclei.

Direct delivery of synthetic expression cassette compositions in vivo will generally be accomplished with or without viral vectors, as described above, by injection using either a conventional syringe, needless devices such as Bioject™ or a gene gun, such as the Accell™ gene delivery system (PowderMed Ltd, Oxford, England).

D. Pharmaceutical Compositions

MARK inhibitors (e.g., CagA peptides, polypeptides, or nucleic acids encoding them) can be formulated into pharmaceutical compositions optionally comprising one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerine, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

A composition of the invention can also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the CagA peptide, polypeptide, or nucleic acid, or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the MARK inhibitor (e.g., when contained in a drug delivery system) in the composition will vary depending on a number of factors, but will optimally be a therapeutically effective dose when the composition is in a unit dosage form or container (e.g., a vial). A therapeutically effective dose can be determined experimentally by repeated administration of increasing amounts of the composition in order to determine which amount produces a clinically desired endpoint.

The amount of any individual excipient in the composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, N.J. (1998), and Kibbe, A. H., Handbook of Pharmaceutical Excipients, 3rd Edition, American Pharmaceutical Association, Washington, D.C., 2000.

The compositions encompass all types of formulations and in particular those that are suited for injection, e.g., powders or lyophilates that can be reconstituted with a solvent prior to use, as well as ready for injection solutions or suspensions, dry insoluble compositions for combination with a vehicle prior to use, and emulsions and liquid concentrates for dilution prior to administration. Examples of suitable diluents for reconstituting solid compositions prior to injection include bacteriostatic water for injection, dextrose 5% in water, phosphate buffered saline, Ringer's solution, saline, sterile water, deionized water, and combinations thereof. With respect to liquid pharmaceutical compositions, solutions and suspensions are envisioned. Additional preferred compositions include those for oral, ocular, or localized delivery.

The pharmaceutical preparations herein can also be housed in a syringe, an implantation device, or the like, depending upon the intended mode of delivery and use. Preferably, the compositions comprising one or more MARK inhibitors (e.g., CagA peptides, polypeptides, or nucleic acids) described herein are in unit dosage form, meaning an amount of a conjugate or composition of the invention appropriate for a single dose, in a premeasured or pre-packaged form.

The compositions herein may optionally include one or more additional agents, such as other drugs for treating Alzheimer's disease or other tauopathies, or other medications used to treat a subject for a condition or disease. Particularly preferred are compounded preparations including a at least one MARK inhibitor (e.g., CagA peptide, polypeptide, or nucleic acid) and one or more drugs for treating Alzheimer's disease or other tauopathies, such as cholinesterase inhibitors (e.g., Razadyne® (galantamine), Exelon® (rivastigmine), Aricept® (donepezil), and Cognex® (tacrine)), N-methyl D-aspartate (NMDA) antagonists (e.g., Namenda® (memantine)), and small molecule inhibitors of MARK, including pyrazolo(1,5-a)pyrimidine and aminothiazole derivatives (see, e.g., International patent applications WO2009152027, WO2007141571, WO2010083145, and WO2010017046, herein incorporated by reference in their entireties). Alternatively, such agents can be contained in a separate composition from the composition comprising a MARK inhibitor (e.g., CagA peptide, polypeptide, or nucleic acid) and co-administered concurrently, before, or after the composition comprising a MARK inhibitor of the invention.

E. Administration

At least one therapeutically effective cycle of treatment with a MARK inhibitor (e.g., a CagA peptide, polypeptide, or a nucleic acid encoding a CagA peptide or polypeptide) will be administered to a subject for treatment of a tauopathy. Tauopathies include, but are not limited to, Alzheimer's disease, progressive supranuclear palsy, dementia pugilistica (chronic traumatic encephalopathy), frontotemporal dementia, frontotemporal lobar degeneration, parkinsonism linked to chromosome 17, lytico-Bodig disease (Parkinson-dementia complex of Guam), ganglioglioma, gangliocytoma, meningioangiomatosis, subacute sclerosing panencephalitis, lead encephalopathy, tuberous sclerosis, Hallervorden-Spatz disease, lipofuscinosis, Pick's disease, corticobasal degeneration, Argyrophilic grain disease (AGD), and tangle-predominant dementia, with NFTs similar to AD, but without plaques.

By “therapeutically effective cycle of treatment” is intended a cycle of treatment that when administered, brings about a positive therapeutic response with respect to treatment of an individual for Alzheimer's disease or other tauopathy. Of particular interest is a cycle of treatment with a MARK inhibitor that reduces tau or PSD-95 hyperphosphorylation. Additionally, a cycle of treatment may reduce or prevent deposition of neurofibrillary tangles, loss of expression of synaptic markers, loss of dendritic spines, or loss of synapses. By “positive therapeutic response” is intended that the individual undergoing treatment according to the invention exhibits an improvement in one or more symptoms of Alzheimer's disease or other tauopathy, including such improvements as reduced deposition of neurofibrillary tangles or improved memory and cognitive function.

In certain embodiments, multiple therapeutically effective doses of compositions comprising one or more MARK inhibitors (e.g., CagA peptides or polypeptides or nucleic acids encoding CagA peptides or polypeptides), and/or one or more other therapeutic agents, such as other MARK inhibitors, drugs for treating Alzheimer's disease or other tauopathies, or other medications will be administered. The compositions of the present invention are typically, although not necessarily, administered orally, via injection (subcutaneously, intravenously, or intramuscularly), by infusion, or locally. Additional modes of administration are also contemplated, such as intracerebral, intraneural, intraspinal, intralesion, intraparenchymatous, pulmonary, rectal, transdermal, transmucosal, intrathecal, pericardial, intra-arterial, intraocular, intraperitoneal, and so forth. In particular embodiments, compositions are administered into the brain or spinal cord of a subject.

The preparations according to the invention are also suitable for local treatment. In a particular embodiment, a composition of the invention is used for localized delivery of a MARK inhibitor, for example, for the treatment of Alzheimer's disease and other tauopathies. For example, compositions may be administered directly into a neuron or by stereotactic injection into the brain. The particular preparation and appropriate method of administration are chosen to target MARK and the site of aberrant tau phosphorylation and neurofibrillary tangle deposition.

The pharmaceutical preparation can be in the form of a liquid solution or suspension immediately prior to administration, but may also take another form such as a syrup, cream, ointment, tablet, capsule, powder, gel, matrix, suppository, or the like. The pharmaceutical compositions comprising one or more MARK inhibitors and other agents may be administered using the same or different routes of administration in accordance with any medically acceptable method known in the art.

In another embodiment, the pharmaceutical compositions comprising one or more MARK inhibitors and/or other agents are administered prophylactically, e.g., to prevent deposition of neurofibrillary tangles. Such prophylactic uses will be of particular value for subjects with symptoms of dementia or pre-existing tau hyperphosphorylation, or who have a genetic predisposition to developing Alzheimer's disease or other tauopathy.

In another embodiment of the invention, the pharmaceutical compositions comprising one or more MARK inhibitors and/or other agents are in a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition.

The invention also provides a method for administering a conjugate comprising a MARK inhibitor as provided herein to a patient suffering from a condition that is responsive to treatment with a MARK inhibitor contained in the conjugate or composition. The method comprises administering, via any of the herein described modes, a therapeutically effective amount of the conjugate or drug delivery system, preferably provided as part of a pharmaceutical composition. The method of administering may be used to treat any condition that is responsive to treatment with a MARK inhibitor. More specifically, the compositions herein are effective in treating Alzheimer's disease and other tauopathies.

Those of ordinary skill in the art will appreciate which conditions a specific MARK inhibitor can effectively treat. The actual dose to be administered will vary depending upon the age, weight, and general condition of the subject as well as the severity of the condition being treated, the judgment of the health care professional, and conjugate being administered. Therapeutically effective amounts can be determined by those skilled in the art, and will be adjusted to the particular requirements of each particular case.

Generally, a therapeutically effective amount will range from about 0.50 mg to 5 grams of a MARK inhibitor daily, more preferably from about 5 mg to 2 grams daily, even more preferably from about 7 mg to 1.5 grams daily. Preferably, such doses are in the range of 10-600 mg four times a day (QID), 200-500 mg QID, 25-600 mg three times a day (TID), 25-50 mg TID, 50-100 mg TID, 50-200 mg TID, 300-600 mg TID, 200-400 mg TID, 200-600 mg TID, 100 to 700 mg twice daily (BID), 100-600 mg BID, 200-500 mg BID, or 200-300 mg BID. The amount of compound administered will depend on the potency of the specific MARK inhibitor and the magnitude or effect on MARK inhibition desired and the route of administration.

A purified MARK inhibitor (again, preferably provided as part of a pharmaceutical preparation) can be administered alone or in combination with one or more other therapeutic agents, such as cholinesterase inhibitors (e.g., Razadyne® (galantamine), Exelon® (rivastigmine), Aricept® (donepezil), and Cognex® (tacrine)), N-methyl D-aspartate (NMDA) antagonists (e.g., Namenda® (memantine)), and small molecule inhibitors of MARK (e.g., pyrazolo(1,5-a)pyrimidine and aminothiazole derivatives), or other medications used to treat a particular condition or disease according to a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Preferred compositions are those requiring dosing no more than once a day.

A MARK inhibitor can be administered prior to, concurrent with, or subsequent to other agents. If provided at the same time as other agents, one or more MARK inhibitors can be provided in the same or in a different composition. Thus, one or more MARK inhibitors and other agents can be presented to the individual by way of concurrent therapy. By “concurrent therapy” is intended administration to a subject such that the therapeutic effect of the combination of the substances is caused in the subject undergoing therapy. For example, concurrent therapy may be achieved by administering a dose of a pharmaceutical composition comprising MARK inhibitor and a dose of a pharmaceutical composition comprising at least one other agent, such as another MARK inhibitor or drug for treating Alzheimer's disease or other tauopathy, which in combination comprise a therapeutically effective dose, according to a particular dosing regimen. Similarly, one or more MARK inhibitors and one or more other therapeutic agents can be administered in at least one therapeutic dose. Administration of the separate pharmaceutical compositions can be performed simultaneously or at different times (i.e., sequentially, in either order, on the same day, or on different days), so long as the therapeutic effect of the combination of these substances is caused in the subject undergoing therapy.

F. Kits

The invention also provides kits comprising one or more containers holding compositions comprising at least one MARK inhibitor (e.g., a CagA peptide or polypeptide or nucleic acid encoding a CagA peptide or polypeptide), and optionally one or more other MARK inhibitors or drugs for treating Alzheimer's disease or other tauopathies. Compositions can be in liquid form or can be lyophilized, as can individual peptides, polypeptides, or nucleic acids. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle).

The kit can further comprise a second container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery devices. The delivery device may be pre-filled with the compositions.

The kit can also comprise a package insert containing written instructions for methods of treating Alzheimer's disease or other tauopathies. The package insert can be an unapproved draft package insert or can be a package insert approved by the Food and Drug Administration (FDA) or other regulatory body.

### III. Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### A Critical Role for the PAR-1/MARK-Tau Axis in Mediating the Toxic Effects of Aβ on Synapses and Dendritic Spines

In this study, we describe a pharmacological approach using a specific peptide inhibitor to probe the importance of PAR-1/MARK in mediating the synaptic and dendritic toxicity of Aβ in primary cultures of rat hippocampal neurons. Our results provide compelling evidence that PAR-1/MARK kinases are critically involved in mediating the synaptic toxicity of Aβ by promoting the aberrant phosphorylation of tau and PSD-95, and that inhibition of PAR-1/MARK kinases represents a viable therapeutic approach.

Materials and Methods

Hippocampal Neuronal Culture and Transfection

Rat E18 hippocampal neuron primary cultures were prepared as described (Yu et al. (2011) Hum. Mol. Genet. 20:3227-3240, herein incorporated by reference in its entirety). Cultured neurons, grown for 10-days in vitro in 12-well plates, were transfected with 2-3 μg of plasmid per well using a CalPhos Mammalian Transfection Kit (Clontech, San Jose, Calif., USA). Fluorescence immunocytochemistry was performed 3 days after transfection. Preparation and concentration of lentiviral particles were performed as described before (Yu et al., supra). Viral transfection of hippocampal neurons was carried out at 10 days after in vitro culture. The next day, the culture medium was replaced with 50% fresh medium and 50% conditioned medium.

Aβ Oligomer Preparation

Commercial Aβ-42 peptide was purchased from rPeptide (Bogart, Ga., USA). Aβ oligomer was prepared following a published protocol (Knowles et al. (2009) J. Neurosci. 29:10627-10637). Briefly, Aβ-42 was dissolved in hexafluoroisopropanol (HFIP) at 4 μg/μl and aliquoted into 0.6 ml tubes. Peptide films were obtained by evaporating HFIP with a Speed VAC and stored at −80° C. Prior to use, the peptide was dissolved in phosphate buffer (pH 7.4) to 80 μM and incubated at 4° C. for 24 hours. Culture medium containing 5 μM Aβ was used to treat neurons.

Recombinant DNA Construction

EGFP-tagged PSD-95-WT in the FHUGW lentivirus vector was described before (Xu et al. (2008) Neuron 57:248-262). The S561A mutation was introduced by site-directed mutagenesis. Tau S2A mutations were introduced as described before (Nishimura et al. Cell, 116:671-682), and subcloned into FHUGW with a C-terminal HA tag. EGFP- and HA-tagged human MARK4 constructs were obtained from Dr. Gerard Drewes. MKI cDNA was obtained from Dr. C. Erec Stebbins, and subcloned into FHUGW vector with a C-terminal EGFP tag.

Immunofluorescence Analysis

Chicken anti-EGFP (1:4000) and rabbit anti-HA (1:3000) antibodies were purchased from Abcam (Cambridge, Mass., USA); mouse anti-Myc (1:1500, 4A6) and mouse anti-PSD-95 (1:300) from Millipore (Billerica, Mass., USA); rabbit anti-GluR1 (1:200) from Calbiochem (San Diego, Calif., USA) and rabbit anti-Synapsin I (1:1000) from Sigma (St Louis, Mo., USA). Immunofluorescence analysis was done essentially as described before (Yu et al., supra). Confocal images were obtained using a Leica TCS SP5 confocal microscope or Zeiss inverted LSM510 confocal microscope (Carl Zeiss, Inc.) with 63×NA 1.4 objectives. Processed confocal images were used to make 2D projections. Dendritic spine number and clusters of PSD-95, GluR1 or Synapsin I were manually counted as the number along a 10 μm length of dendrite and presented as mean±SEM. Each experiment was done at least three times and six to nine neurons were randomly selected for analysis each time. Between 15 and 30 dendritic processes were selected for quantification. Data were analyzed while blind to the conditions.

Electrophysiology in Dissociated Cultures

Whole-cell patch-clamp recordings were made from neurons at 13-14 DIV in voltage clamp mode using a Multiclamp 700B amplifier (Molecular Devices, Union City, Calif., USA), digitized at 10 kHz and filtered at 4 kHz. Data were acquired and analyzed using AxographX (Axograph, Sydney). Whole-cell recording pipettes (3-5 MΩ) were filled with a solution containing 135 mM CsMeSO₄, 8 mM NaCl, 10 mM HEPES, 0.25 mM EGTA, 2 mM Mg₂ATP, 0.3 mM Na₃GTP, 0.1 mM spermine and 7 mM phospho-creatine. The bath solution contained: 140 mM NaCl, 2.5 mM KCl, 2 mM CaCl₂, 2 mM MgCl₂, 10 mM HEPES-NaOH pH 7.4 and 10 mM glucose. Miniature AMPAR-mediated EPSCs were isolated by including D-APV (50 μM), picrotoxin (100 μM) and 1 μM tetrodotoxin in the bath solution. All recordings were performed at a holding potential of −70 mV at room temperature. mEPSCs were identified using a template with a threshold of −6 pA (2.5×SD of the noise) and were individually proofread for accuracy. To plot summary graphs, the average frequency from the EGFP control cells from each culture preparation was normalized. Individual cells from all four conditions were then compared with this normalized average.

Results

Overexpression of MARK4 Induces Defects in Synapses and Dendritic Spines

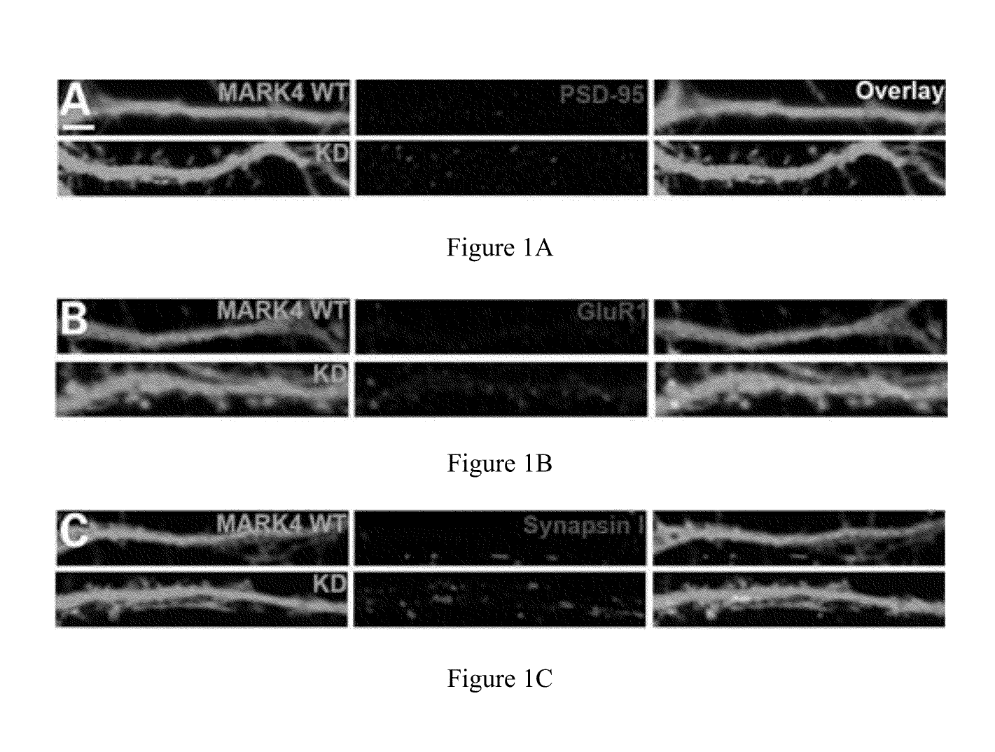

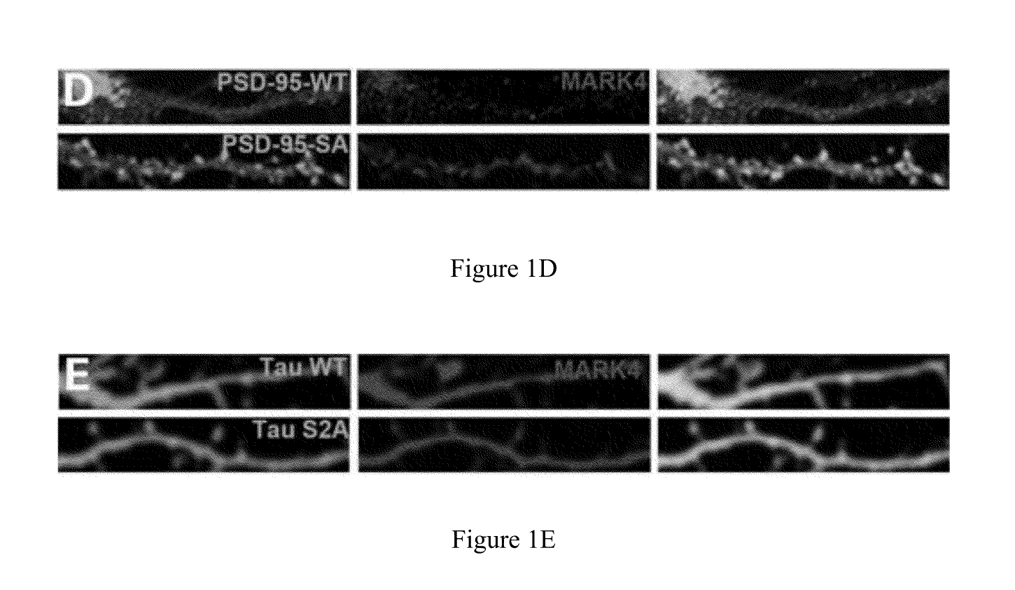

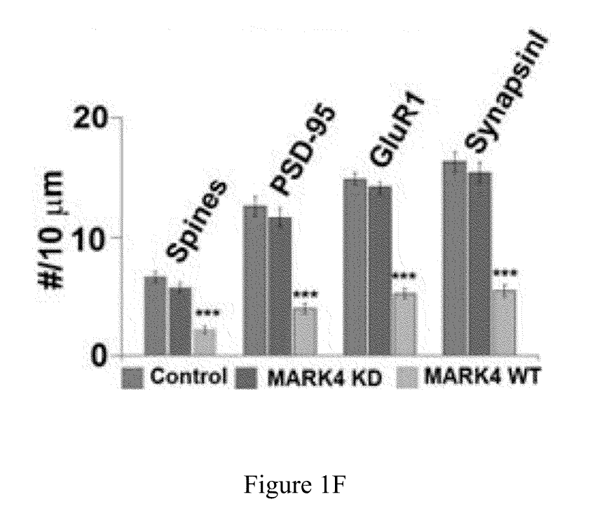

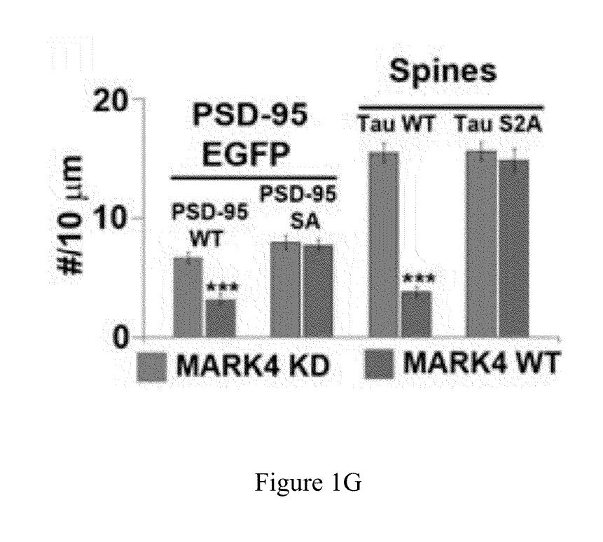

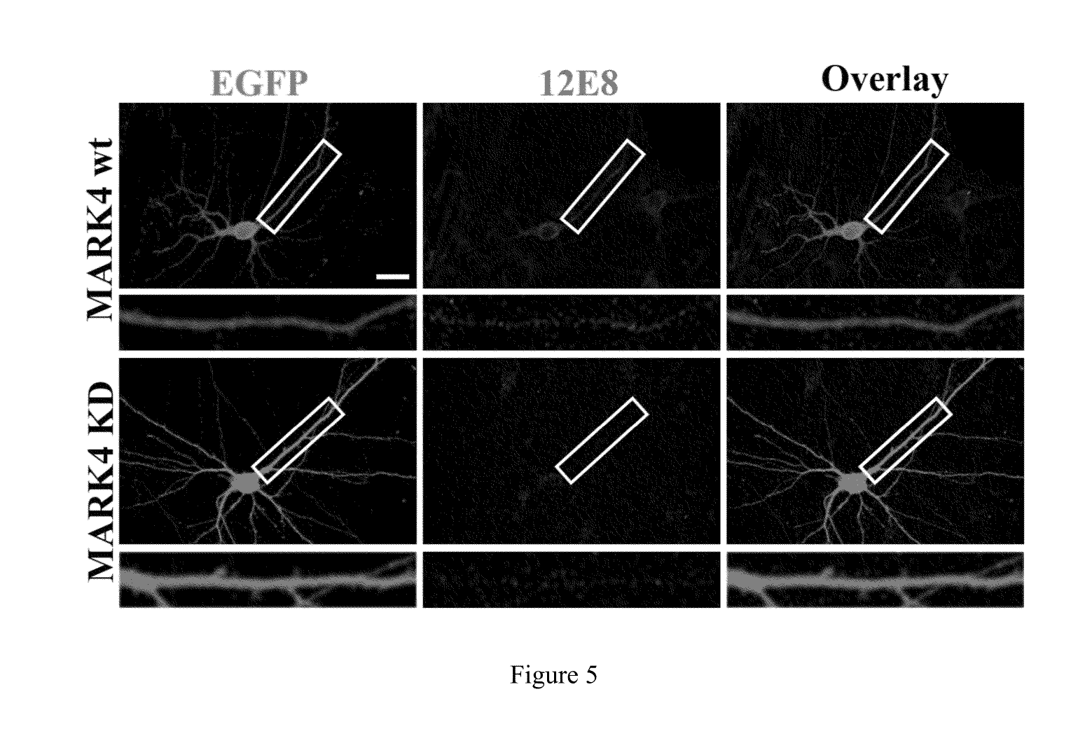

We used cultured rat primary hippocampal neurons to test whether the PAR-1/MARK family kinases mediate AD-related neurotoxicity in mammals. In addition to being the relevant cell type for studying AD pathogenesis, these neurons offer excellent cellular resolution for studying synaptic and dendritic spine morphologies and for electrophysiological characterization. As expected, we found that overexpression of Drosophila PAR-1 or mammalian MARK4 (Trinczek et al. (2004) J. Biol. Chem. 279:5915-5923) in hippocampal neurons resulted in hyperphosphorylation of tau at the phosphorylation sites recognized by the 12E8 antibody (FIG. 5), sites known to be phosphorylated by PAR-1/MARK (Drewes et al. (1997) Cell 89:297-308; Nishimura et al. (2004) Cell 116:671-682). This was accompanied by a number of phenotypes including loss of dendritic spines (FIGS. 1A-C and 1F), the delocalization of PSD-95 from synapses (FIGS. 1A and 1F) and decreased expression of other synaptic markers such as GluR1, a subunit of o-amino-3-hydroxy-5-methyl-4-isoxazolepropionic acid (AMPA) receptors (FIGS. 1B and 1F), and the presynaptic protein Synapsin I (FIGS. 1C and 1F). In addition, we also observed defects in mitochondria localization in PAR-1 or MARK4 overexpressing neurons (data not shown). The kinase-dead form of MARK4 (MARK4-KD) had no such effect, indicating that kinase activity is required for inducing the observed toxic effects. Both MARK4-WT and MARK4-KD exhibited relatively uniform distribution in the dendrites (FIGS. 1A-1C).

Previous studies in Drosophila showed that PAR-1 could regulate the postsynaptic localization of Dlg through direct phosphorylation (Zhang et al. (2007) Neuron 53:201-215). The phosphorylation site is conserved in PSD-95, the mammalian homolog of Dlg. To test whether MARK4 may directly act on PSD-95 to regulate its synaptic localization, we generated a mutant form of PSD-95 in which the conserved PAR-1/MARK target site is mutated to Ala (PSD-95-SA). Both the WT and SA mutant forms of PSD-95 were fused with GFP to facilitate visualization. In the same expression vector, shRNAs targeting endogenous PSD-95 but not the exogenous PSD-95-GFP were also expressed (Xu et al. (2008) Neuron, 57:248-262). Thus, the observed effects were largely free of potential interference from endogenous PSD-95. Although PSD-95-WT-EGFP was delocalized from the dendritic spines by MARK4 and was present uniformly in the dendritic shaft, PSD-95-SA-EGFP effectively resisted the effect of MARK4 and maintained its punctate synaptic localization (FIGS. 1D and 1G), consistent with MARK4 directly regulating PSD-95 localization in mammalian hippocampal neurons through phosphorylation.

Expression of a Non-Phosphorylatable Form of Tau Blocks PAR-1/MARK Toxicity

We were interested in identifying the key substrates through which PAR-1/MARK overexpression leads to synaptic and dendritic spine toxicity. Previous studies showed that PAR-1 overexpression in Drosophila photoreceptors caused neurotoxicity through phosphorylation of tau in the microtubule-binding domain, and that a mutant form of tau that had the PAR-1 site mutated (tauS2A) was not only itself non-toxic, but also, could block PAR-1 overexpression-induced neurotoxicity (Nishimura (2004) Cell 116: 671-682). Co-transfection of a similar phosphorylation-mutant form of tau (h-tau-SA) blocked the toxic effects of MARK4 in rat hippocampal neurons, including the spine loss, whereas wild-type human tau (h-tau-WT) failed to do so (FIGS. 1E and 1G). This is consistent with tau being a major mediator of MARK4 toxicity on synapses and dendritic spines.

Expression of Non-Phosphorylatable Form of Tau Blocks Aβ Toxicity

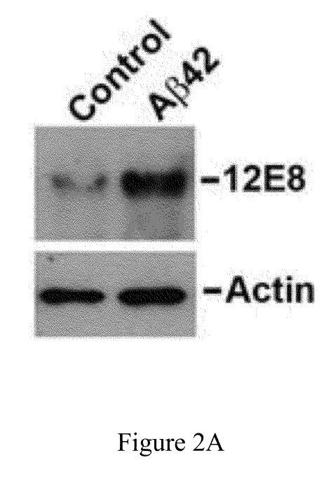

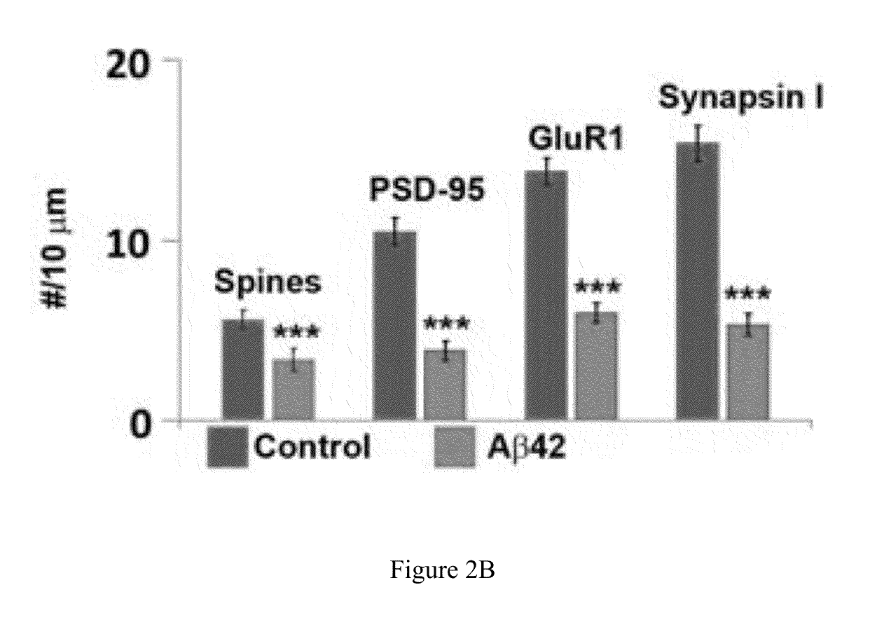

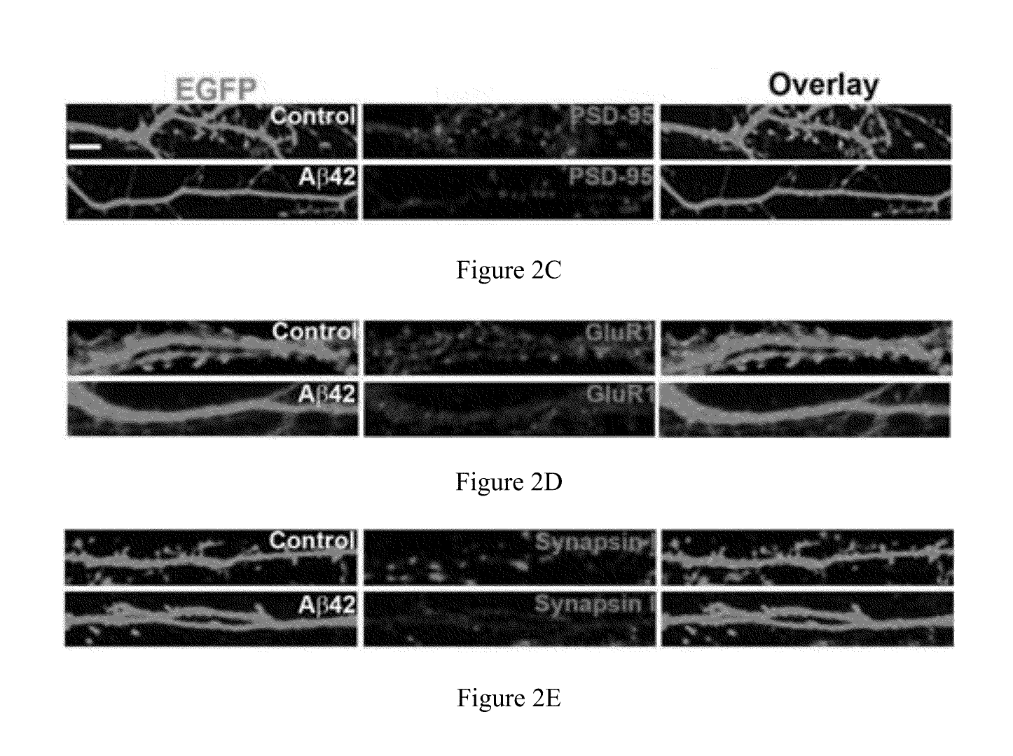

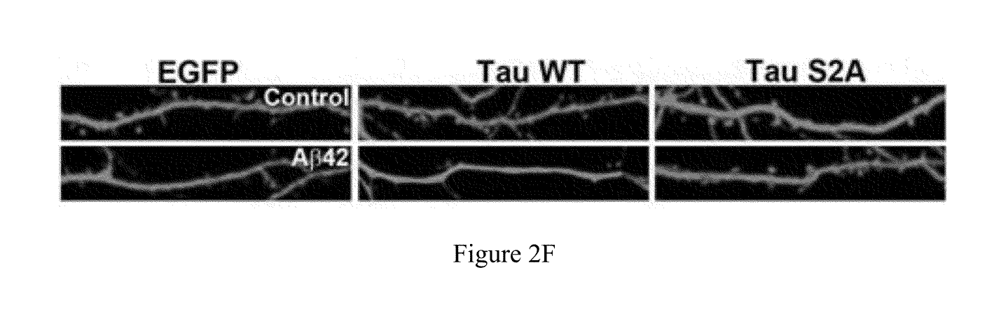

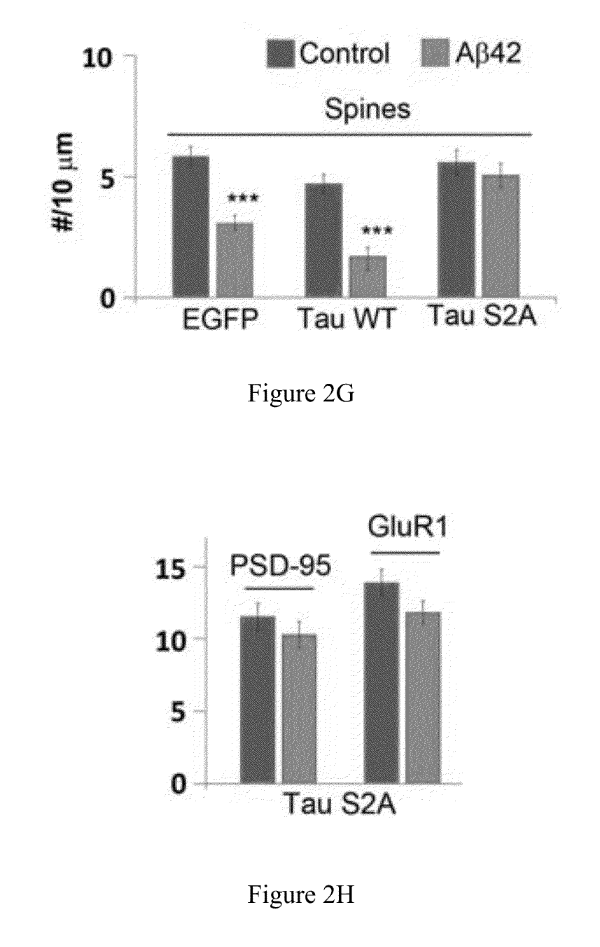

We further tested whether phosphorylation of tau by MARK might mediate the toxicity of Aβ on neuronal synapses and dendritic spines. Although MARK activation and elevation of tau phosphorylation at PAR-1/MARK target sites occurred in hippocampal neurons after Aβ oligomer treatment (Jin et al. (2011) Proc. Natl. Acad. Sci. USA 108:5819-5824; Zempel et al. (2010) J. Neurosci. 30:11938-11950), it is not clear whether these events were disease-causing or simply a compensatory, protective response. Indeed, overexpression of MARK2 was reported to be able to rescue tau-induced synapse and spine loss (Thies et al. (2007) J. Neurosci. 27:2896-2907). Consistent with previous reports (Jin et al., supra; Zempel et al., supra), treatment of rat hippocampal neurons with synthetic Aβ, prepared using a well-characterized procedure that enriches for Aβ oligomers (Knowles et al. (2009) J. Neurosci. 29:10627-10637), resulted in increased tau phosphorylation at the 12E8 sites (FIG. 2A), suggesting that Aβ treatment had activated MARK kinases. Increased phosphorylation of tau at a site recognized by the PHF-1 phospho-tau antibody was also observed (data not shown). Aβ treatment also caused losses of synaptic marker expression and dendritic spines as seen in the PAR-1/MARK overexpression condition (FIGS. 2B-2E and 2G). Strikingly, in neurons transfected with h-tau-S2A but not h-tau-WT, the toxic effect of Aβ in causing spine loss was ameliorated (FIGS. 2F and 2G). The toxic effects of Aβ on the density of synaptic marker PSD-95 and GluR1 clusters were also rescued by h-tau-S2A (FIG. 2H). These results support the notion that phosphorylation of tau by PAR-1/MARK family kinases is a primary downstream event by which Aβ exerts its toxicity on hippocampal synapses and dendritic spines.

A PAR-1/MARK Inhibitor Effectively Blocked Aβ Toxicity

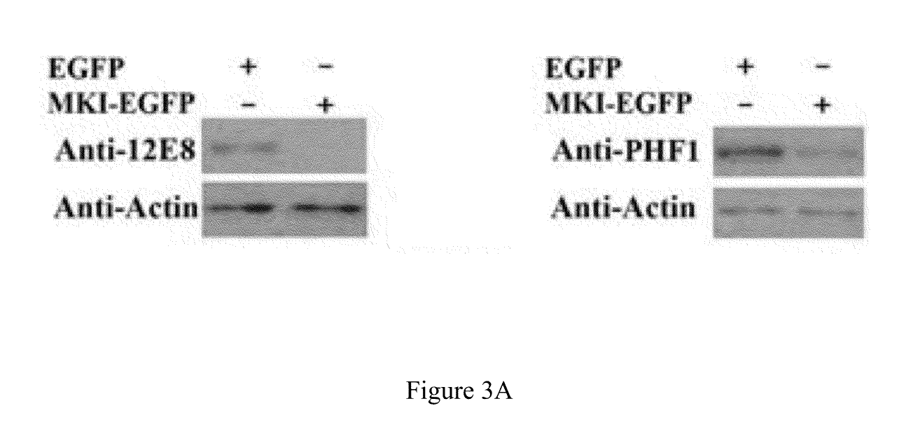

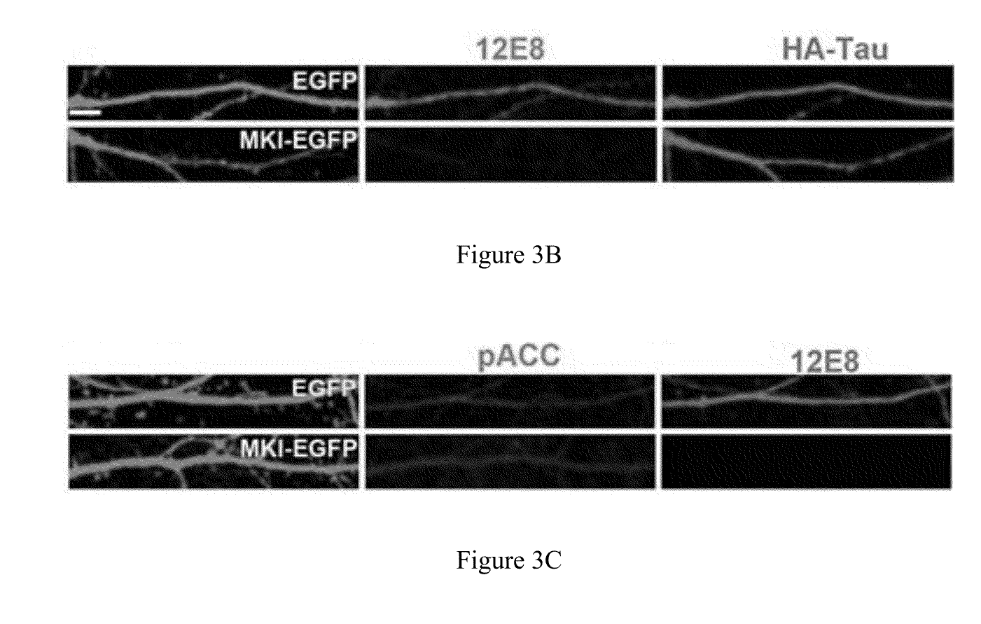

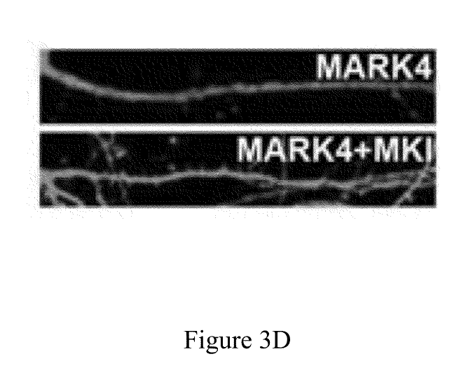

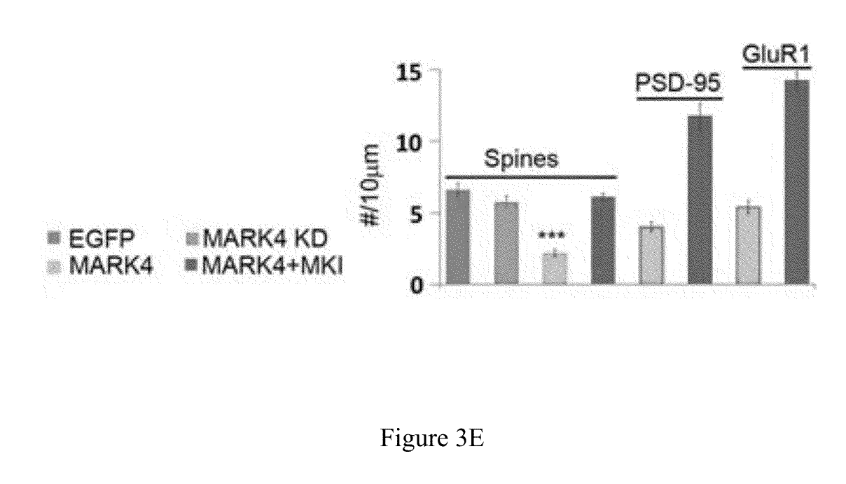

Next we wished to assess the involvement of endogenous MARK kinases in mediating Aβ toxicity. To circumvent potential functional redundancy among at least four mammalian MARK family members, we used a peptide inhibitor (MKI, SEQ ID NO:1) derived from the CagA protein of Helicobacter pylori, which can specifically bind with high affinity to the substrate-binding sites of all MARKs by mimicking the natural substrates of these kinases (Nesic et al. (2010) Nat. Struct. Mol. Biol. 17:130-132). We first confirmed that expression of an MKI-GFP fusion protein in rat hippocampal neurons effectively attenuated MARK4-mediated phosphorylation of both endogenous tau (FIG. 3A) and transfected human tau at the 12E8 sites (FIG. 3B). Phosphorylation of tau at the PHF-1 site was also reduced by MKI (FIG. 3A). It is possible that the PHF-1 site is also targeted by PAR-1/MARKs in vivo, or that the phosphorylation of tau at the 12E8 sites is a prerequisite for PHF-1 site phosphorylation, as the 12E8 sites were previously shown to be required for tau phosphorylation at other sites (Nishimura (2004) Cell 116:671-682). Supporting the specificity of the MKI effect on PAR-1/MARK, under similar experimental conditions, MKI-EGFP did not affect the phosphorylation of acetyl-CoA carboxylase (ACC) by AMP-activated kinase (AMPK) (FIG. 3C), which is closely related to PAR-1/MARK on the protein kinase phylogenetic tree (Lizcano et al. (2004) EMBO J. 23:833-843). MARK4 overexpression-induced synaptic defects including spine loss (FIGS. 3D and 3E) and reduction of PSD-95 and GluR1 puncta density were all effectively blocked by MKI-EGFP (FIG. 3E).

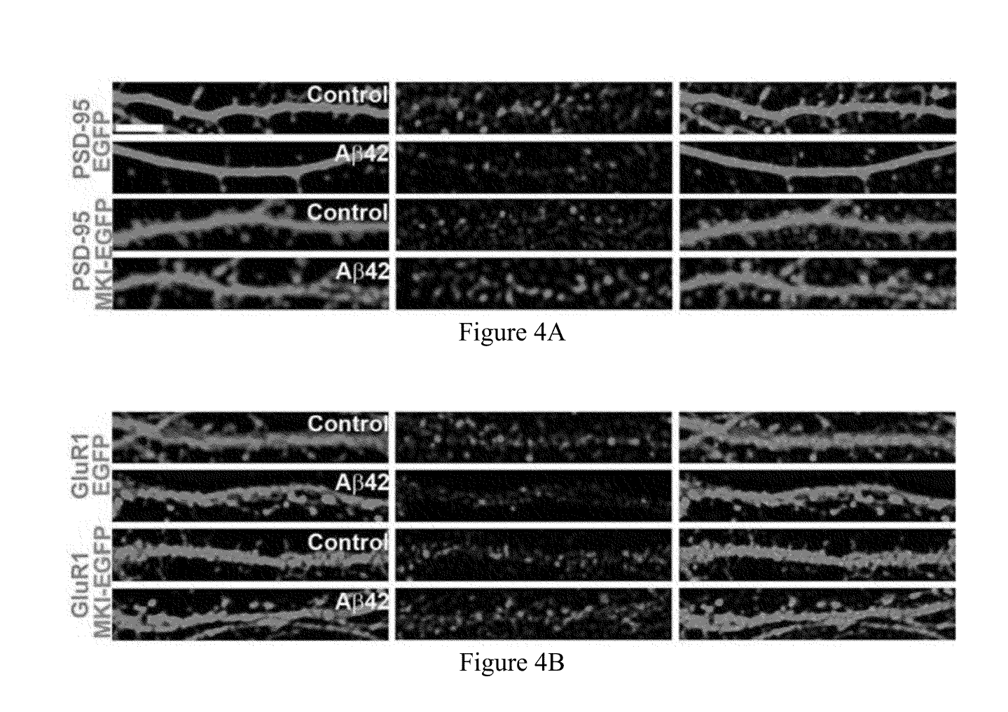

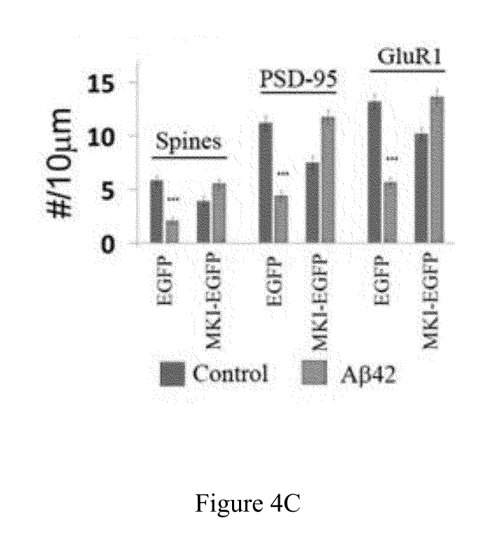

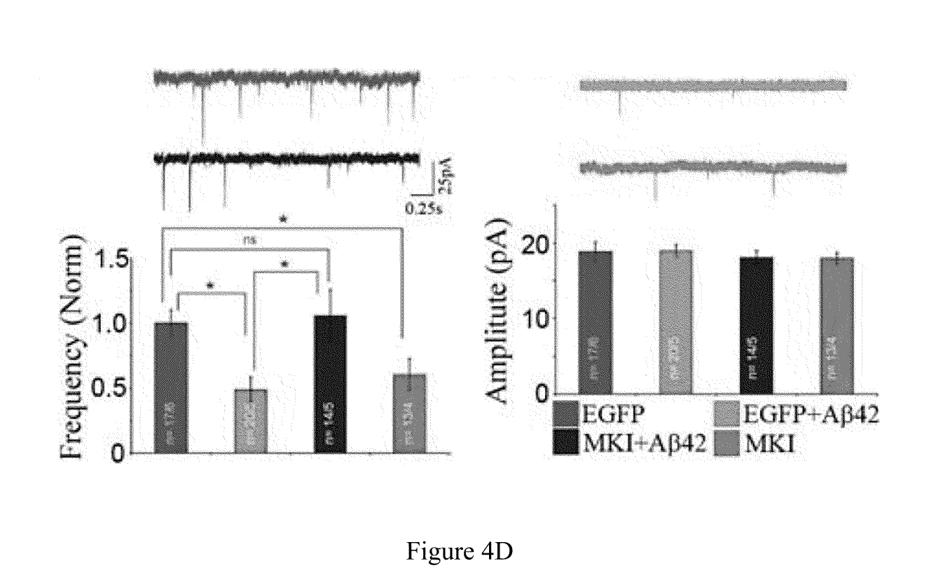

To test the function of MARK kinases in mediating the toxicity of Aβ on synapses and dendritic spines, we next examine the effect of MKI in modulating the toxicity of Aβ. Remarkably, MKI-GFP expression effectively blocked the morphological defects caused by Aβ, such as the loss of dendritic spines (FIGS. 4A-4C), and the reduction in the density of PSD-95 (FIGS. 4A and 4C) and GluR1 puncta (FIGS. 4B and 4C).

We further examined the effect of MKI-EGFP at the functional level by performing electrophysiological analysis. Aβ treatment caused a reduction in the frequency but not in the amplitude of AMPAR-mediated miniature excitatory post-synaptic current (mEPSC) (FIG. 4D). The reduction in the mEPSC frequency suggested that Aβ is causing a reduction in synapse number, whereas the unchanged mEPSC amplitude indicated that the postsynaptic AMPA receptor composition and/or activity of the remaining synapses were probably not affected. The reduction of mEPSC frequency caused by Aβ was effectively rescued by MKI-EGFP (FIG. 4D). We note that neurons expressing MKI-EGFP alone also showed reduced mEPSC frequency but not amplitude (FIG. 4D), suggesting that the PAR-1/MARK family kinases have a normal physiological function in hippocampal neurons and that its appropriate level is important for synaptic function.

Discussion

The amyloid and tau lesions are pathological hallmarks of AD that are present in virtually all AD cases. The relationship between the two, however, remains unresolved. Accumulating evidence supports the toxic oligomers formed by the Aβ peptide as the disease-causing agents, and synapses and dendritic spines as their major sites of action. Here we show that Aβ acts through the PAR-1/MARK family kinases to impinge on tau to affect synaptic function and dendritic spine morphogenesis. This conclusion is supported by the suppression of Aβ toxicity by either the co-expression of a phospho-mutant form of tau that can no longer be phosphorylated by PAR-1/MARK, or the specific inhibition of PAR-1/MARK with a peptide inhibitor. Our findings are consistent with the hypothesis that tau abnormality is a major downstream pathogenic event in the signaling cascade initiated by Aβ (Hardy et al. (2002) Science 297:353-356), and the observation that removal of endogenous tau attenuates learning and memory deficits in AD models (Roberson et al. (2007) Science 316:750-754).

One major question concerns how tau abnormality, specifically its hyperphosphorylation, leads to synaptic and dendritic spine pathology. Despite tau being best known as an axonal protein, phospho-tau was shown to accumulate in dendritic spines, where it may affect the synaptic trafficking and/or anchoring of glutamate receptors, thereby influencing postsynaptic function (Hoover et al. (2010) Neuron 68:1067-1081). It is not clear whether phospho-tau is being actively transported to the dendritic compartment, or that due to its compromised ability to bind microtubules, especially after being phosphorylated by PAR-1/MARK, phospho-tau simply diffuses from axon to other cellular compartments. Dendritic tau was recently found to recruit fyn kinase, facilitating fyn-mediated phosphorylation of GluR2 and stabilizing the interaction between GluR2 and PSD-95 (Ittner et al. (2010) Cell, 142:387-397). This potentially promotes excitotoxicity and represents a possible mechanism by which dendritic tau mediates Aβ toxicity. It remains to be determined whether dendritic tau has a normal physiological function. Since Aβ-induced spine loss was preventable by taxol (Zempel et al. (2010) J. Neurosci. 30:11938-11950), tau-related microtubule destabilization and trafficking defects may also mediate Aβ toxicity.

Much of the molecular events linking Aβ to tau pathologies remain to be elucidated. The implication of PAR-1/MARK in this process offers a new entry point to further dissect the signaling process. Several kinases have been shown to act upstream of PAR-1/MARK (Matenia et al. Trend. Biochem. Sci. 34:332-342), including LKB1 (Wang et al. (2007) J. Neurosci. 27:574-581). It would be interesting to test the involvement of these kinases in mediating Aβ toxicity through the regulation of PAR-1/MARK. Previous studies have indicated that tau phosphorylation at the PAR-1/MARK target sites also responds to other stress stimuli including osmotic stress, oxidative stress, serum deprivation and glutamate-induced excitotoxicity (Wang et al., supra; Zempel et al., supra), suggesting that other triggers of the disease may also impinge on the PAR-1/MARK-tau axis. Aβ could also induce reversible synapse loss by modulating an NMDA-type GluR-dependent signaling pathway (Shankar et al. (2007) J. Neurosci. 27:2866-2875), or use long-term depression-related signaling mechanisms to affect synaptic function and dendritic spine morphology (Hsieh et al. (2006) Neuron 52:831-843). Whether these signaling pathways are connected to the PAR-1/MARK-tau axis will be important future research directions. It also remains to be determined whether Aβ oligomers act by directly activating transmembrane receptors, changing neuronal membrane properties or, simply, causing cellular stress (Reddy et al. (2008) Trend. Mol. Med. 14:45-53; Zempel et al., supra).

Our results suggest that targeting PAR-1/MARK kinases represents a potential strategy to prevent the synaptic toxicity of Aβ, thereby preventing or treating the synaptic defects of AD. One major concern is the potential side effects, given the myriad cellular functions of PAR-1/MARK kinases (Matenia et al. (2009) Trend. Biochem. Sci. 34:332-342). This concern is warranted by our observation that although MKI restored normal neuro-transmission to Aβ oligomer-treated neurons, it led to reduced mEPSC frequency in mock-treated neurons, suggesting that appropriate levels of PAR-1/MARK activity is important for neurotransmission. Thus, in Aβ oligomer-treated neurons, which possess elevated PAR-1/MARK activity (Zempel et al., supra), MKI tuned down kinase activity to a level compatible with normal neurotransmission. However, in normal neurons, reduction of PAR-1/MARK activity by MKI to below-threshold levels could compromise neurotransmission. It will therefore be necessary to adjust MKI dosages to achieve therapeutic effect without causing serious side effects. The same could be said for therapeutic approaches targeting Aβ or tau. For example, the production of Aβ is positively regulated by neuronal activity (Bero et al. (2011) Nat. Neurosci. 14:750-756), and Aβ can in turn depress excitatory synaptic transmission (Kamenetz et al. (2003) Neuron 37:925-937). This negative feedback regulatory loop may normally serve to restrain neuronal hyperactivity, such that therapeutic approaches leading to too much removal of Aβ might have unwanted side effects as well.

### Example 2

### MKI Formulations

MKI Formulation 1

In preparation of the first formulation, the MKI peptide was dissolved in 10 mM phosphate buffer saline (Invitrogen) containing 5% bovine serum albumin (BSA) to a final peptide concentration of 1%. A clear solution was obtained, which was spray dried using a vibrating mesh spray method and a hydrophobic surface treated particle collector. By this method, spherical particles ranging from 500 nm to 5 μm in diameter were obtained.

MKI Formulation 2

In preparation of the second formulation, the MKI peptide was dissolved in 100% 1,1,1,3,3,3-hexafluoro-2-propanol (HFIP) by vortexing followed by sonication for half an hour. A clear solution was obtained with an effective peptide concentration of 4%. This solution was then diluted with 100% ethanol (1:1) to produce a milky solution having an effective peptide concentration of 2%. This solution, referred to as solution A, contained no precipitate and did not become transparent after 15 minutes of sonication. Solution B was prepared by dissolving distearyl-phosphatidylcholine (DSPC) and dipalmitoylphosphatidylcholine (DPPC) in 100% ethanol to final concentrations of 2% each. Solution B was sonicated for half an hour. Solutions A and B were mixed (1:1) to generate the formulation. The final effective concentrations of the MKI peptide, DSPC and DPPC in this solution were 1% each. This solution, though milky, contained no precipitate. This solution was spray dried using a vibrating mesh spray method and a hydrophobic surface treated particle collector and characterized by scanning electron microscope (SEM) analysis.

While the preferred embodiments of the invention have been illustrated and described, it will be appreciated that various changes can be made therein without departing from the spirit and scope of the invention.

What is claimed is:

1. A method of treating a subject for a tauopathy, the method comprising administering a therapeutically effective amount of an inhibitor of microtubule affinity-regulating kinase (MARK) to the subject.

2. The method of claim 1, wherein the inhibitor is selected from the group consisting of:
a) a peptide comprising the amino acid sequence of SEQ ID NO: 1;
b) a peptide comprising an amino acid sequence having at least 95% identity to the sequence of SEQ ID NO: 1, wherein the peptide inhibits the activity of MARK;
c) a polypeptide comprising the amino acid sequence of SEQ ID NO: 2;
d) a polypeptide comprising an amino acid sequence having at least 95% identity to the sequence of SEQ ID NO: 2, wherein the polypeptide inhibits the activity of MARK;
e) a polypeptide comprising the amino acid sequence of SEQ ID NO: 4;
f) a polypeptide comprising an amino acid sequence having at least 95% identity to the sequence of SEQ ID NO: 4, wherein the polypeptide inhibits the activity of MARK; and
g) a CagA peptide of 14 to 50 amino acids in length comprising at least residues 941 to 954 of the amino acid sequence of SEQ ID NO:4.

3. The method of claim 2, wherein the inhibitor is a peptide consisting of the amino acid sequence of SEQ ID NO:1.

4. The method of claim 1, wherein the inhibitor is a recombinant polynucleotide comprising a promoter operably linked to a polynucleotide encoding an inhibitor of MARK, wherein the polynucleotide encoding the inhibitor of MARK is selected from the group consisting of:
a) a polynucleotide encoding a peptide comprising the amino acid sequence of SEQ ID NO: 1;
b) a polynucleotide encoding a peptide comprising an amino acid sequence having at least 95% identity to the sequence of SEQ ID NO: 1, wherein the peptide inhibits the activity of MARK;
c) a polynucleotide encoding a polypeptide comprising the amino acid sequence of SEQ ID NO: 2;
d) a polynucleotide encoding a polypeptide comprising an amino acid sequence having at least 95% identity to the sequence of SEQ ID NO: 2, wherein the polypeptide inhibits the activity of MARK;
e) a polynucleotide encoding a polypeptide comprising the amino acid sequence of SEQ ID NO: 4;
f) a polynucleotide encoding a polypeptide comprising an amino acid sequence having at least 95% identity to the sequence of SEQ ID NO: 4, wherein the polypeptide inhibits the activity of MARK;
g) a polynucleotide encoding a CagA peptide of 14 to 50 amino acids in length, said peptide comprising at least residues 941 to 954 of the amino acid sequence of SEQ ID NO:4;
h) a CagA polynucleotide of 42 to 150 nucleotides in length comprising at least nucleotides 2821 to 2862 of the nucleotide sequence of SEQ ID NO:3;
i) a polynucleotide comprising the nucleotide sequence of SEQ ID NO:5; and
j) a polynucleotide comprising a nucleotide sequence having at least 95% identity to the sequence of SEQ ID NO:5, wherein the polynucleotide encodes a polypeptide that inhibits the activity of MARK.

5. The method of claim 4, wherein the polynucleotide encodes a peptide consisting of the amino acid sequence of SEQ ID NO:1.

6. The method of claim 4, wherein the polynucleotide comprises the nucleotide sequence of SEQ ID NO:5.

7. The method of claim 4, wherein the recombinant polynucleotide comprises a lentivirus expression vector.

8. The method of claim 1, wherein the tauopathy is Alzheimer's disease.

9. A method of inhibiting tau hyperphosphorylation in the brain of a subject, the method comprising administering an effective amount of an inhibitor of microtubule affinity-regulating kinase (MARK) to the subject.

10. The method of claim 9, wherein the inhibitor is selected from the group consisting of:
a) a peptide comprising the amino acid sequence of SEQ ID NO: 1;
b) a peptide comprising an amino acid sequence having at least 95% identity to the sequence of SEQ ID NO: 1, wherein the peptide inhibits the activity of MARK;
c) a polypeptide comprising the amino acid sequence of SEQ ID NO: 2;
d) a polypeptide comprising an amino acid sequence having at least 95% identity to the sequence of SEQ ID NO: 2, wherein the polypeptide inhibits the activity of MARK;
e) a polypeptide comprising the amino acid sequence of SEQ ID NO: 4;
f) a polypeptide comprising an amino acid sequence having at least 95% identity to the sequence of SEQ ID NO: 4, wherein the polypeptide inhibits the activity of MARK; and
g) a CagA peptide of 14 to 50 amino acids in length comprising at least residues 941 to 954 of the amino acid sequence of SEQ ID NO:4.

11. The method of claim 10, wherein the inhibitor is a peptide consisting of the amino acid sequence of SEQ ID NO:1.

12. The method of claim 9, wherein the inhibitor is a recombinant polynucleotide comprising a promoter operably linked to a polynucleotide encoding an inhibitor of MARK, wherein the polynucleotide encoding the inhibitor of MARK is selected from the group consisting of:
a) a polynucleotide encoding a peptide comprising the amino acid sequence of SEQ ID NO: 1;
b) a polynucleotide encoding a peptide comprising an amino acid sequence having at least 95% identity to the sequence of SEQ ID NO: 1, wherein the peptide inhibits the activity of MARK;
c) a polynucleotide encoding a polypeptide comprising the amino acid sequence of SEQ ID NO: 2;
d) a polynucleotide encoding a polypeptide comprising an amino acid sequence having at least 95% identity to the sequence of SEQ ID NO: 2, wherein the polypeptide inhibits the activity of MARK;
e) a polynucleotide encoding a polypeptide comprising the amino acid sequence of SEQ ID NO: 4;
f) a polynucleotide encoding a polypeptide comprising an amino acid sequence having at least 95% identity to the sequence of SEQ ID NO: 4, wherein the polypeptide inhibits the activity of MARK;
g) a polynucleotide encoding a CagA peptide of 14 to 50 amino acids in length, said peptide comprising at least residues 941 to 954 of the amino acid sequence of SEQ ID NO:4;
h) a CagA polynucleotide of 42 to 150 nucleotides in length comprising at least nucleotides 2821 to 2862 of the nucleotide sequence of SEQ ID NO:3;
i) a polynucleotide comprising the nucleotide sequence of SEQ ID NO:5; and
j) a polynucleotide comprising a nucleotide sequence having at least 95% identity to the sequence of SEQ ID NO:5, wherein the polynucleotide encodes a polypeptide that inhibits the activity of MARK.

13. The method of claim 12, wherein the polynucleotide encodes a peptide consisting of the amino acid sequence of SEQ ID NO:1.

14. The method of claim 12, wherein the recombinant polynucleotide comprises a lentivirus expression vector.

15. The method of claim 9, wherein hyperphosphorylation of PSD-95 is reduced in the presence of the inhibitor compared to the hyperphosphorylation of PSD-95 in the absence of the inhibitor.

16. The method of claim 9, wherein loss of expression of synaptic markers is reduced in the presence of the inhibitor compared to the loss of expression of synaptic markers in the absence of the inhibitor.

17. The method of claim 9, wherein loss of dendritic spines is reduced in the presence of the inhibitor compared to the loss of dendritic spines in the absence of the inhibitor.

18. The method of claim 9, wherein loss of synapses is reduced in the presence of the inhibitor compared to the loss of synapses in the absence of the inhibitor.

19. A kit comprising a pharmaceutical composition comprising a MARK inhibitor and instructions for treating a tauopathy.

20. The kit of claim 19, further comprising means for delivering said composition to a subject.

21. The kit of claim 19, wherein the MARK inhibitor is selected from the group consisting of:
a) a peptide comprising the amino acid sequence of SEQ ID NO: 1;
b) a peptide comprising an amino acid sequence having at least 95% identity to the sequence of SEQ ID NO: 1, wherein the peptide inhibits the activity of MARK;
c) a polypeptide comprising the amino acid sequence of SEQ ID NO: 2;
d) a polypeptide comprising an amino acid sequence having at least 95% identity to the sequence of SEQ ID NO: 2, wherein the polypeptide inhibits the activity of MARK;
e) a polypeptide comprising the amino acid sequence of SEQ ID NO: 4;
f) a polypeptide comprising an amino acid sequence having at least 95% identity to the sequence of SEQ ID NO: 4, wherein the polypeptide inhibits the activity of MARK; and
g) a CagA peptide of 14 to 50 amino acids in length comprising at least residues 941 to 954 of the amino acid sequence of SEQ ID NO:4.

22. The kit of claim 19, wherein the MARK inhibitor is a recombinant polynucleotide comprising a promoter operably linked to a polynucleotide selected from the group consisting of:
a) a polynucleotide encoding a peptide comprising the amino acid sequence of SEQ ID NO: 1;
b) a polynucleotide encoding a peptide comprising an amino acid sequence having at least 95% identity to the sequence of SEQ ID NO: 1, wherein the peptide inhibits the activity of MARK;
c) a polynucleotide encoding a polypeptide comprising the amino acid sequence of SEQ ID NO: 2;
d) a polynucleotide encoding a polypeptide comprising an amino acid sequence having at least 95% identity to the sequence of SEQ ID NO: 2, wherein the polypeptide inhibits the activity of MARK;
e) a polynucleotide encoding a polypeptide comprising the amino acid sequence of SEQ ID NO: 4;
f) a polynucleotide encoding a polypeptide comprising an amino acid sequence having at least 95% identity to the sequence of SEQ ID NO: 4, wherein the polypeptide inhibits the activity of MARK;
g) a polynucleotide encoding a CagA peptide of 14 to 50 amino acids in length, said peptide comprising at least residues 941 to 954 of the amino acid sequence of SEQ ID NO:4;
h) a CagA polynucleotide of 42 to 150 nucleotides in length comprising at least nucleotides 2821 to 2862 of the nucleotide sequence of SEQ ID NO:3;
i) a polynucleotide comprising the nucleotide sequence of SEQ ID NO:5; and
j) a polynucleotide comprising a nucleotide sequence having at least 95% identity to the sequence of SEQ ID NO:5, wherein the polynucleotide encodes a polypeptide that inhibits the activity of MARK.

23. A method of preparing a pharmaceutical composition comprising a MARK inhibitor, the method comprising dissolving the MARK inhibitor in a pharmaceutically acceptable solvent and spray drying to produce a powder, wherein the powder comprises spherical particles ranging from about 500 nm to about 5 μm in diameter.

24. The method of claim 23, wherein the MARK inhibitor is selected from the group consisting of:
a) a peptide comprising the amino acid sequence of SEQ ID NO: 1; and
b) a peptide comprising an amino acid sequence having at least 95% identity to the sequence of SEQ ID NO: 1, wherein the peptide inhibits the activity of MARK.

25. A pharmaceutical composition prepared according to the method of claim 23.
